SEQUENCE LISTING (16 sequences)

SEQ ID NO: 1 - DNA, 20 bp, synthetic construct
  source 1..20: mol_type = other DNA
cttagttggtggagcgattt

SEQ ID NO: 2 - DNA, 18 bp, synthetic construct
  source 1..18: mol_type = other DNA
gctgaacgccacttgtcc

SEQ ID NO: 3 - DNA, 22 bp, synthetic construct
  source 1..22: mol_type = other DNA
gcagcactacttcttgaccacc

SEQ ID NO: 4 - DNA, 22 bp, synthetic construct
  source 1..22: mol_type = other DNA
tctgctcctgaggattgacgtc

SEQ ID NO: 5 - DNA, 21 bp, synthetic construct
  source 1..21: mol_type = other DNA
ctcttctgcctgctgcacttg

SEQ ID NO: 6 - DNA, 22 bp, synthetic construct
  source 1..22: mol_type = other DNA
atgggctacaggcttgtcactc

SEQ ID NO: 7 - DNA, 23 bp, synthetic construct
  source 1..23: mol_type = other DNA
catcactgccacccagaagactg

SEQ ID NO: 8 - DNA, 23 bp, synthetic construct
  source 1..23: mol_type = other DNA
atgccagtgagcttcccgttcag

SEQ ID NO: 9 - DNA, 22 bp, synthetic construct
  source 1..22: mol_type = other DNA
ttggtgaagggactgagctgtt

SEQ ID NO: 10 - DNA, 19 bp, synthetic construct
  source 1..19: mol_type = other DNA
acttccaggggcaagccat

SEQ ID NO: 11 - DNA, 21 bp, synthetic construct
  source 1..21: mol_type = other DNA
cgcctgagtggctgtcttttg

SEQ ID NO: 12 - DNA, 19 bp, synthetic construct
  source 1..19: mol_type = other DNA
tggggctgatcccgttgat

SEQ ID NO: 13 - DNA, 21 bp, synthetic construct
  source 1..21: mol_type = other DNA
gagtgacaagcctgtagccca

SEQ ID NO: 14 - DNA, 20 bp, synthetic construct
  source 1..20: mol_type = other DNA
ttgagatccatgccgttggc

SEQ ID NO: 15 - DNA, 23 bp, synthetic construct
  source 1..23: mol_type = other DNA
agacaaagccagagtccttcaga

SEQ ID NO: 16 - DNA, 22 bp, synthetic construct
  source 1..22: mol_type = other DNA
aggagagcattggaaattgggg

(19) United States
(12) Patent Application Publication
(10) Pub. No.: US 2026/0034240 A1
(43) Pub. Date: Feb. 5, 2026

(54) MULTIFUNCTIONAL CREAM HYDROGELS FOR POSTOPERATIVE ADHESION PREVENTION

(71) Applicant: Board of Regents of the University of Nebraska, Lincoln, NE (US)

(72) Inventors: Bin Duan, Omaha, NE (US); Mark Carlson, Omaha, NE (US); Bo Liu, Omaha, NE (US)

(21) Appl. No.: 18/998,804

(22) PCT Filed: Aug. 15, 2023
(86) PCT No.: PCT/US2023/072186

§ 371 Date: Jan. 27, 2025

Related U.S. Application Data
(60) Provisional application No. 63/397,934, filed on Aug. 15, 2022.

(51) Int. Cl. A61K 47/69; A61K 45/06; A61K 47/54; A61K 47/61

(52) U.S. Cl. CPC A61K 47/6903; A61K 45/06; A61K 47/54; A61K 47/545; A61K 47/61

(57) ABSTRACT

Multifunctional hydrogels and methods of use thereof are provided.

This application claims priority under 35 U.S.C. § 119 (e) to U.S. Provisional Patent Application No. 63/397,934, filed Aug. 15, 2022. The foregoing application is incorporated by reference herein.

### FIELD OF THE INVENTION

This application relates to the field of postoperative adhesions. More specifically, this invention provides compositions and methods for inhibiting or preventing adhesion formation, particularly for abdominal adhesion formation.

### BACKGROUND OF THE INVENTION

Several publications and patent documents are cited throughout the specification in order to describe the state of the art to which this invention pertains. Each of these citations is incorporated herein by reference as though set forth in full.

Postoperative abdominal adhesions occur in 90% of patients after an abdominal operation and can produce complications (Krielen, et al. (2020) Lancet 395 (10217): 33-41; Stapleton, et al. (2019) Nat. Biomed. Engr., 3 (8): 611-620; Tabibian, et al. (2017) Ann. Med. Surg. (Lond) 15:9-13). Each year in the USA, about 300,000 patients who have had a previous abdominal operation undergo another abdominal operation, representing a several billion USD financial burden. The presence of adhesions from the previous operation increases the difficulty and risk of the next operation (Zindel, et al. (2021) Science 371 (6533): 993; Sikirica, et al. (2011) BMC Surgery 11:13). Abdominal adhesions can produce serious clinical problems, including intestinal obstructions, chronic pelvic pain, female sterility, and even death (Chandel, et al. (2021) Macromol. Biosci. 21 (3): 2000395; Park, et al. (2020) Materials (Basel) 13 (14): 3056; Raissier, et al. (2022) Amer. J. Obstetrics Gynecol. 226 (3): S1296-S1297). The lifetime risk of an adhesive small bowel obstruction (SBO) after an abdominal surgical procedure is 12-16% (Tong, et al. (2020) Acute Med. Surg., 7 (1): e587). There are some methods to minimize adhesion formation using delicate and minimally invasive surgery, such as laparoscopic surgery (Krielen, et al. (2020) Lancet 395 (10217): 33-41). However, these methods cannot completely eliminate adhesion formation.

Two major postoperative abdominal antiadhesion treatments have been evaluated: pharmacological treatments and physical barrier materials. Local or systemic anti-inflammatory drugs and anticoagulants (e.g., aspirin, dexamethasone, and heparin) have not been effective in preventing abdominal adhesion (Chandel, et al. (2021) Macromol. Biosci. 21 (3): 2000395; Fatehi Hassanabad, et al. (2021) Biomolecules 11 (7): 1027). Barrier materials have been more convenient and effective as antiadhesion treatments (Chandel, et al. (2021) Macromol. Biosci. 21 (3): 2000395). However, the commercially available film barriers (e.g., Interceed® and Seprafilm®) have suffered from handling and low efficacy issues (Chandel, et al. (2021) Macromol. Biosci., 21 (3): 2000395; Rajab, et al. (2010) J. Surg Res., 161 (2): 246-249). These commercial film-based products have a relatively short retention time and do not guarantee the complete coverage of the wounds with irregular surfaces. Most importantly, the films tend to adhere to wet surfaces, severely restricting their use during surgical procedures. The icodextrin solution (Adept®) also has suffered from low efficacy in abdominal adhesion prevention, as most of the liquid quickly drains from the treated surface (Rajab, et al. (2010) J. Surg Res., 161 (2): 246-249; Brown, et al. (2007) Fertil. Steril., 88 (5): 1413-1426). Therefore, better therapies are needed to decrease the incidence and severity of postoperative tissue adhesions.

By combining the advantages of film and liquid barriers, the injectable hydrogel can perfectly cover irregular wounds and has potential in abdominal adhesion inhibition after open or laparoscopic surgery (Wu, et al. (2022) Adv. Funct. Mater., 32:2110066). Many types of injectable hydrogels with excellent antifouling adhesive capabilities, such as carboxyl-containing dynamically cross-linked supramolecular polymer-nanoparticle hydrogels (Stapleton, et al. (2019) Nat. Biomed. Engr., 3 (8): 611-620), Janus hydrogels (Cui, et al. (2020) Adv. Funct. Mater., 30 (49): 2005689; Liang, et al. (2022) Adv. Mater., 34:2108992), photocurable catechol-grafted hyaluronic acid (HA) hydrogels (Wu, et al. (2022) Adv. Funct. Mater., 32:2110066; Zeng, et al. (2022) Acta Biomater., 151:210), hotmelt tissue adhesives (Nishiguchi, et al. (2022) Acta Biomater., 146:80), bottlebrush inspired injectable hydrogels (Gao, et al. (2022) Bioactive Materials 16:27), cellulose-based thermo-gels (Sultana, et al. (2020) Carbohydr. Polym., 229:115552), and zwitterionic hydrogels (Guo, et al. (2020) Chem. Mater., 32 (15): 6347-6357; Zhang, et al. (2021) Adv. Funct Mater., 31 (10): 20094), were prepared by using natural or synthetic polymers through chemical modification or crosslinking. Although efficacy has been shown, these hydrogel systems encounter at least some of the following limitations: (1) rapid degradation rate and short retention time; (2) presence of toxic residues of cross-linking agents; (3) the use of ultraviolet irradiation; (4) lack of self-healing or self-fused ability to prevent fragmentation during the injection process; and (5) slow hydrogel gelation. Significantly, most of these hydrogels mainly serve as physical barriers to separate the injured tissues and prevent the postsurgical adhesions, which generally provide limited benefits, since they cannot reduce local oxidative stress and inflammatory responses.

After an abdominal operation, a large number of inflammatory cells are locally activated with an associated oxidative stress to the mesothelium (Zindel, et al. (2021) Science 371 (6533): 993; Louwe, et al. (2021) Nat. Commun., 12 (1): 1770; Zhang, et al. (2019) J. Exp Med., 216 (6): 1291-1300). Excessive reactive oxygen species (ROS), such as hydrogen peroxide and superoxide anions, are produced under oxidative stress, are cytotoxic, and can promote the formation of adhesions (Tang, et al. (2020) Acta Biomater., 116:84-104; Nakagawa, et al. (2015) Biomaterials 69:165-173). In addition, excessive coagulation and inflammatory responses disrupt the balance between fibrin formation and fibrinolysis systems, resulting in the formation of adhesions between tissues and organs (Tang, et al. (2020) Acta Biomater., 116:84-104). To improve barrier material performance against postoperative adhesions, several studies have incorporated antioxidative or anti-inflammatory properties into these materials (Nakagawa, et al. (2015) Biomaterials 69:165-173; Wang, et al. (2020) ACS Nano 14 (7): 8202-8219), including the integration of functional molecules Tempol (4-hydroxy-2,2,6,6-tetramethylpiperidine-1-oxyl) and phenylboronic acid pinacol ester conjugated with β-cylcodextrin (Wang, et al. (2020) ACS Nano 14 (7): 8202-8219; Li, et al. (2018) Adv. Sci. (Weinh) 5 (10): 1800781), mitomycin C¹⁹, and quercetin (Zeng, et al. (2022) Acta Biomater., 151:210).

Despite these pioneering advances in this field, the rational design of new hydrogels with a combination of antioxidative, anti-inflammatory, and antiadhesive properties is still needed.

### SUMMARY OF THE INVENTION

In accordance with the instant invention, hydrogels are provided. In certain embodiments, the hydrogel comprises carbohydrate polymer comprising carboxyl groups, a pyrogallol-containing compound, phenylboronic acid or a phenylboronic acid-containing compound, and a hydrogel forming polymer. In certain embodiments, the carbohydrate polymer comprising carboxyl groups is hyaluronic acid. In certain embodiments, the carbohydrate polymer comprising carboxyl groups (e.g., hyaluronic acid), pyrogallol-containing compound, and phenylboronic acid or a phenylboronic acid-containing compound form a microgel. In certain embodiments, the pyrogallol-containing compound is linked or conjugated to the phenylboronic acid or phenylboronic acid-containing compound. In certain embodiments, the carbohydrate polymer comprising carboxyl groups (e.g., hyaluronic acid) is conjugated or linked to the phenylboronic acid or a phenylboronic acid-containing compound. In certain embodiments, the carbohydrate polymer comprising carboxyl groups (e.g., hyaluronic acid) is conjugated or linked to the hydrogel forming polymer via the phenylboronic acid or a phenylboronic acid-containing compound conjugated or linked to the carbohydrate polymer comprising carboxyl groups (e.g., hyaluronic acid). In certain embodiments, the pyrogallol-containing compound is linked or conjugated to the phenylboronic acid or phenylboronic acid-containing compound via a boronic ester bond. In certain embodiments, the carbohydrate polymer comprising carboxyl groups (e.g., hyaluronic acid) is conjugated or linked to the hydrogel forming polymer via the phenylboronic acid or a phenylboronic acid-containing compound via a boronic ester bond. In certain embodiments, the carbohydrate polymer comprising carboxyl groups (e.g., hyaluronic acid) is conjugated or linked to the phenylboronic acid or a phenylboronic acid-containing compound via an amide bond. In certain embodiments, the pyrogallol-containing compound is a flavonoid. In certain embodiments, the pyrogallol-containing compound is a catechin. In certain embodiments, the pyrogallol-containing compound is epigallocatechin gallate (EGCG). In certain embodiments, the phenylboronic acid-containing compound comprises phenylboronic acid linked to a functional group such as —NH₂ via a linker such as a C₁-C₃ alkyl linker. In certain embodiments, the phenylboronic acid-containing compound is (3-aminomethylphenyl) boronic acid. In certain embodiments, hydrogel forming polymer comprises polyvinyl alcohol. In certain embodiments, the hydrogel comprises epigallocatechin gallate, hyaluronic acid, phenylboronic acid, and polyvinyl alcohol. In certain embodiments, the carbohydrate polymer comprising carboxyl groups (e.g., hyaluronic acid) is further conjugated or linked to a zwitterionic monomer. In certain embodiments, the zwitterionic monomer is N,N-dimethyl-N-(2-acryloylethyl)-N-(3-sulfopropyl) ammonium betaine (SPDA). In certain embodiments, hydrogel further comprises a therapeutic agent and/or cell.

In accordance with another aspect of the instant invention, compositions comprising a hydrogel of the instant invention and a pharmaceutically acceptable carrier are provided.

In accordance with another aspect of the instant invention, devices, particularly implantable devices, coated with a hydrogel of the instant invention are provided.

In accordance with another aspect of the instant invention, methods of inhibiting and/or preventing post-operation adhesions in a subject in need thereof are provided. In certain embodiments, the method comprises applying or administering a hydrogel of the instant invention to the subject, particularly at a site of surgery or to an injured or damaged tissue. In certain embodiments, the hydrogel is applied or administered topically, by injection, by spraying, or by aerosolization.

In accordance with another aspect of the instant invention, methods of delivering a therapeutic agent and/or cell to a subject in need thereof are provided. In certain embodiments, the hydrogel further comprises the therapeutic agent and/or cell. In certain embodiments, the hydrogel is applied or administered to the subject topically, by injection, by spraying, or by aerosolization. In accordance with another aspect of the instant invention, methods of treating, inhibiting, and/or preventing a microbial or bacterial infection in a subject in need thereof are provided, particularly wherein the hydrogel further comprises an antimicrobial and/or antibiotic. In accordance with another aspect of the instant invention, methods for treating, inhibiting, and/or preventing inflammation in a subject in need thereof are provided, particularly wherein the hydrogel further comprises an anti-inflammatory agent. In accordance with another aspect of the instant invention, methods of treating, inhibiting, and/or preventing cancer in a subject in need thereof are provided, particularly wherein the hydrogel further comprises a chemotherapeutic agent. In accordance with another aspect of the instant invention, methods of treating, inhibiting, and/or preventing diabetes in a subject in need thereof are provided, particularly wherein the hydrogel further comprises an anti-diabetic drug.

In accordance with another aspect of the instant invention, methods of synthesizing or manufacturing a hydrogel of the instant invention are provided.

### BRIEF DESCRIPTION OF THE DRAWINGS

FIGS. 1A-1F show the morphology, size distribution, cytotoxicity, and hemolysis of the HPE microgels. FIGS. 1A and 1B provide representative SEM images, particle size distributions, and mean sizes of HₗPE (FIG. 1A) and HₕPE microgels (FIG. 1B). FIG. 1C provides the zeta potentials of HₗPE and HₕPE, which were −30 and −41 mV, respectively (n=3). FIG. 1D provides the CCK-8 results which show that there was no significant difference in the L929 cell proliferation on days 1 and 3 after various treatments (n=4). FIG. 1E provides Live/Dead™ images L929 cells which show that the cells could grow normally after microgel treatment for 3 days. FIG. 1F provides a graph of hemolysis which shows that the HPE microgel was hemocompatible, with less than 1% hemolysis (n=3). NC: negative control (PBS); PC: positive control (1% Triton™ X-100); ns: no significant difference; n=3; **** p<0.0001. FIG. 1G provides a schematic of HPE, wherein the wavy line represents HA. FIG. 1H provides a schematic of the dynamic crosslinkage of HPE to PVA.

FIG. 2A provides a schematic illustration of a mouse cecum-abdominal wall adhesion model with or without treatment with HPE-PVA hydrogels. FIGS. 2B-2I show the morphology, rheological properties, and biodegradability of HPE-PVA hydrogels. FIG. 2B provides images of HPE microgels and HPE-PVA hydrogels.

FIG. 2C provides representative SEM images showing that the hydrogels exhibit porous structures with microgel particles. FIG. 2D provides images showing the injectability of HPE-PVA with food color dyes through an insulin needle and their self-healing capacity after contacting each other. FIG. 2E provides degradation profiles of the HPE-PVA hydrogels in PBS solution at 37° C. (n=3). FIG. 2F provides time-sweep curves of HPE-PVA hydrogels, showing that the G′ values were dominant over the corresponding G″, indicating a stable, solid-like state (0-5 min, 1 Hz, strain of 10%, 37° C.). FIG. 2G provides frequency-sweep curves (0.1-100 rad/s, under 10% strain, 37° C.) of hydrogels. FIG. 2H provides step-strain measurements of HPE-PVA hydrogels, with high strains (600%) and low strains (10%) to characterize the extent and rate of stationary self-healing (1 Hz, 37° C.). FIG. 2I provides shear-rate sweep curves showing that the hydrogels were highly shear-thinning, reducing their viscosity by more than 2 orders of magnitude over shear rates extending from 0.1 to 100 s⁻¹.

FIGS. 3A-3D provide the cytocompatibility, hemocompatibility, and anti-cell adhesion of HPE-PVA hydrogels. The cytocompatibility of L929 was evaluated through cell proliferation and viability assays using CCK-8 (n=4; ns: no significant difference) (FIG. 3A) and Live/Dead™ imaging on day 3 (FIG. 3B). FIG. 3C provides a graph of the hemolysis results of HPE-PVA hydrogels. NC: negative control (PBS); PC: positive control (1% Triton™ X-100); ns: no significant difference; n=3; **** p<0.001. FIG. 3D shows the cellular attachment of L929 cells on the tissue culture polystyrene (TCPS) surface and HPE-PVA hydrogels after 18 hours of seeding.

FIGS. 4A-4H show the antioxidative and anti-inflammatory effects of HPE-PVA hydrogels. FIG. 4A provides images of the alleviation of oxidative stress in L929 cells as monitored via ROS probe (H2DCFDA) staining after treatments with PBS, H₂O₂, HₗPE-PVA+H₂O₂, and HₕPE-PVA+H₂O₂. FIG. 4B provides a graph of the oxidized H2DCFDA fluorescence signal intensity as quantified by a microplate reader (Ex=488 nm, Em=535 nm) (n=5). FIG. 4C provides a graph of the radical scavenging rate of HPE-PVA by a DPPH assay. The stable free radical species, reactions with the radical scavenger, and the rate of radical scavenging can be measured by the colorimetric change of DPPH at 517 nm of absorbance (n=3). FIG. 4D provides a schematic of the protocol of human monocyte differentiation into macrophages and M1 macrophages. FIGS. 4E and 4F provide the relative gene expressions of TNF-α and NFκB, respectively, in M1 macrophages after the indicated treatments as evaluated by qPCR (n=3; relative gene expression is presented as normalized to 18S and expressed relative to macrophages treated with PBS). FIGS. 4G and 4H provide graphs of the secretion of TNF-α and IL-1β secretions, respectively, in the supernatants by macrophages after various treatments as detected by ELISA (n=4). ns: no significant difference, *p<0.05, ** p<0.01, *** p<0.001, and **** p<0.0001.

FIGS. 5A-5E show the antiadhesive efficiency of HPE-PVA hydrogels in a murine cecum-abdominal wall adhesion model. FIG. 5A provides images of typical abdominal adhesions in the Sham, Injury-only, HₗPE-PVA, HₕPE-PVA, and Seprafilm® groups at POD 14. Dashed circles indicated the adhesion sites. FIG. 5B provides a graph of adhesion scores at POD 14 (n=8). FIGS. 5C and 5D provide images of H&E staining (FIG. 5C) and Masson's trichrome staining (FIG. 5D) of tissue sections in different groups. Ce: cecum; AW: abdominal wall. The arrows are pointed to the adhesion sites. FIG. 5E provides images of gross observation of a typical adhesion of various scores after abdominal surgery. Adhesion tissues were marked with dot circles.

FIGS. 6A-6J show the effects of HPE-PVA hydrogels on oxidative stress and inflammation in vivo. FIGS. 6A-6D provide graphs of the gene expressions of iNOS (FIG. 6A), TGF-β1 (FIG. 6B), TNF-α (FIG. 6C), and IL-6 (FIG. 6D) in different groups at POD 14. FIG. 6E provides images of immunofluorescence staining images of CD68, iNOS, and DAPI in the adhesion tissues (for the groups with adhesions) and cecum tissues (for the group without adhesions). FIG. 6F provides images of DHE and DAPI staining of adhesion tissues (for the groups with adhesions) and cecum tissues (for the group without adhesions). FIGS. 6G and 6H provide graphs of the fluorescent area ratio of iNOS/DAPI (FIG. 6G) and CD68/DAPI (FIG. 6H) (n=3). FIG. 6I provides a graph of the fluorescent area ratio of DHE/DAPI (n=3). FIG. 6J provides an image of the Western blot analysis of TNF-α and IL-1β in the adhesion tissues (for the groups with adhesions) and abdominal wall tissues (for the group without adhesions) at POD 14 (*p<0.05, ** p<0.01, *** p<0.001, and **** p<0.0001).

FIGS. 7A-7H show the effects of HPE-PVA hydrogels on fibrinolytic activity. FIGS. 7A and 7B provide graphs of the gene expressions of tPA (FIG. 7A) and PAI-1 (FIG. 7B) in the adhesion tissues (for the groups with adhesions) and cecum tissues (for the group without adhesions) (n=3). FIG. 7C provides an image of a Western blot analysis for tPA and PAI-1 in each group. FIGS. 7D and 7E provide images of representative immunofluorescence staining of tPA (FIG. 7D) and PAI-1 (FIG. 7E) and DAPI in different groups at POD 14. FIGS. 7F and 7G provide graphs of a semiquantitative analysis of the tPA positive area (FIG. 7F) and PAI-1 positive area (FIG. 7G) relative to the DAPI positive area. FIG. 7H provides images of representative immunohistochemical staining of α-SMA in the intra-abdominal adhesions or the abdominal wall areas from each group at POD 14. The dotted lines in the indicated squares are the adhesion regions. * p<0.05, ** p<0.01, and *** p<0.001; ns: no significant difference.

FIG. 8A provides a graph of the fluorescent area ratio of (S100A8+S100A9)/DAPI (n=3, *p<0.05, ** p<0.01). FIG. 8B provides a representative image of Western blot analysis for S100A8+S100A9 in each group. FIG. 8C provides a graph of the quantitative analysis of S100A8+S100A9 in Sham, Injury-only, HₕPE-PVA hydrogel, and Seprafilm® groups (n=3, *p<0.05, ** p<0.01). FIG. 8D provides IHC images of S100 A8/A9 protein staining in the Sham, injury-only, HₕPE-PVA hydrogel, and Seprafilm® groups. The dot line attributes to the expression of $100 A8/A9. The original magnification ×20.

FIG. 9 provides representative SEM images of the HSPE-PVA zwitterionic hydrogel showing that the hydrogels exhibited porous structures with microgel particles.

FIGS. 10A and 10B provides graphs of the cytotoxicity of PBS (untreated), HPE-PVA, and HSPE-PVA on L929 fibroblast cells (FIG. 10A) and mesothelium cells (FIG. 10B).

FIG. 11 provides confocal microscopy images of L929 fibroblast cells and mesothelium cells grown on TCPS (control), HPE-PVA hydrogel, HSPE-PVA hydrogel.

### DETAILED DESCRIPTION OF THE INVENTION

Postoperative peritoneal adhesion is a common and serious clinical problem after surgery (Moris, et al. (2017) J. Gastrointestinal Surg., 21 (10): 1713-1722). It leads to numerous serious medical complications including intestinal obstruction. chronic pain, female sterility, and even death (Wu, et al. (2017) J. Controlled Rel., 261:318-336). The incidence of recurrent adhesion is 80% or more even if the surgeries are performed by using minimally invasive techniques (Zhang, et al. (2021) Adv. Funct. Mater., 31 (10): 2009431; Zhao, et al. (2021) Chem. Engr. J., 404:127096; Krielen, et al. (2020) Lancet 395 (10217): 33-41). In order to remove the pre-existing postoperative adhesion, adhesiolysis is performed as a standard surgical procedure (Zhang, et al. (2018) Acta Biomaterialia 74:439-453). However, patients may suffer the risk of recurrent adhesion due to the trauma caused by adhesiolysis (Menzies, et al. (1990) Ann. R. Coll. Surg. Engl., 72:60-63; Krielen, et al. (2020) Lancet 395 (10217): 33-41; Brown, et al. (2007) Fertility Sterility 88 (5): 1413-26). The prevention or recurrent adhesion is more challenging because the injury is more severe and the adhesion mechanism is more complicated (Brown, et al. (2007) Fertility Sterility 88 (5): 1413-26).

Current antiadhesive strategies focus mostly on physical barriers and are unsatisfactory and inefficient (Zhang, et al. (2021) Adv. Funct. Mater., 31 (10): 2009431; Zhang, et al. (2020) PNAS 117:32046-32055). Further, few approaches have proven to be effective in preventing adhesion reformation after adhesiolysis.

Herein, advanced sprayable or injectable cream-like hydrogels with multiple functionalities, including rapid gelation, self-healing, antioxidation, anti-inflammation, antifibrosis, and anti-cell adhesion, were designed and synthesized. The multifunctional hydrogels were facilely formed by the conjugation reaction of phenylboronic acid (PBA)-epigallocatechin-3-gallate (EGCG) and hyaluronic acid (HA)-based microgels and poly(vinyl alcohol) (PVA) based on the dynamic boronic ester bond between boronic acid and 1,3-diol groups. The physicochemical properties of the hydrogels including antioxidative and anti-inflammatory activities were systematically characterized. A mouse cecum-abdominal wall adhesion model was implemented to investigate the efficacy of the microgel-based hydrogels in preventing postoperative abdominal adhesions. The hydrogels, with a high molecular weight HA, significantly decreased the inflammation, oxidative stress, and fibrosis and reduced the abdominal adhesion formation, compared to the commercial Seprafilm® group or Injury-only group. Label-free quantitative proteomics analysis demonstrated that S100A8 and S100A9 expressions were associated with adhesion formation and the microgel-containing hydrogels inhibited this expression. The microgel-containing hydrogels with multifunctionality decreased the formation of postoperative intra-abdominal adhesions in a murine model, demonstrating therapeutic effectiveness for clinical applications.

The multifunctional hydrogel can be used to prevent the postoperative peritoneal adhesions and repeated-injury adhesion after adhesiolysis. During the postoperative adhesion formation, inflammatory cells infiltrate around the wound once the peritoneum is damaged. Then, inflammation and coagulation processes are initiated to promote formation of fibrin clots for self-healing (Tang, et al. (2020) Acta Biomaterialia 116:84-104). It is desirable to inhibit excessive fibrin deposition in the damaged peritoneal cavity (Zhang, et al. (2018) Acta Biomaterialia 74:439-453). It is also desirable to reduce contact between organs in the abdominal cavity and the damaged peritoneum by using physical barriers. The cytokines released by platelets, together with the degradation of products or cots, attract additional macrophages, neutrophils, and mesothelial cells (Tang, et al. (2020) Acta Biomaterialia 116:84-104). The peritoneal macrophages in the peritoneal cavity can act like platelets to form superaggregates, resulting in abdominal adhesion (Herrick, et al. (2021) Science 371 (6533); Zindel, et al. (2021) Science 371 (6533): eabe0595). The hydrogels provided herein can reduce or eliminate contact between injured cecum and apposing parietal peritoneal wall by physical barrier, modulate inflammatory response, and/or downregulate fibrin deposition.

In accordance with the instant invention, hydrogels are provided, particularly for the inhibition (e.g., repress, hinder, or make occur less frequently) and/or prevention of post-operative adhesions, particularly abdominal adhesions. In certain embodiments, the hydrogel is a macromolecular polymer gel including a network. In certain embodiments, the hydrogel is a polymer matrix able to retain water in a swollen state. In certain embodiments, the hydrogel comprises microgels. In certain embodiments, the hydrogels of the instant invention are biocompatible. In certain embodiments, the hydrogels of the instant invention are biodegradable. In certain embodiments, the hydrogels of the instant invention comprise microgels (e.g., HA-PBA-EGCG) crosslinked with a hydrogel forming polymer (e.g., PVA).

In certain embodiments, the hydrogel comprises a carbohydrate polymer (e.g., polysaccharide) with carboxyl groups or a carbohydrate polymer that can be modified with carboxyl groups (e.g., carboxylated). Examples of suitable carbohydrate polymers include, without limitation: cellulose (e.g., carboxylated cellulose), heparin, carboxylated dextran, and hyaluronic acid, particularly heparin, carboxylated dextran, and hyaluronic acid. In certain embodiments, the carbohydrate polymer comprising carboxyl groups is hyaluronic acid. For simplicity, the carbohydrate polymer is generally referred to herein as hyaluronic acid. However, the instant invention encompasses the replacement of hyaluronic acid with a carbohydrate polymer with carboxyl groups or a carbohydrate polymer that can be modified with carboxyl groups (e.g., carboxylated).

In certain embodiments, the hydrogel comprises a complex or compound comprising hyaluronic acid conjugated to a pyrogallol-containing compound via phenylboronic acid or a phenylboronic acid-containing compound. In certain embodiments, the pyrogallol-containing compound is linked or conjugated to the phenylboronic acid or phenylboronic acid-containing compound via a boronic ester bond. In certain embodiments, the hyaluronic acid is conjugated or linked to the phenylboronic acid or phenylboronic acid-containing compound by an amide bond. In certain embodiments, the hyaluronic acid is further conjugated or linked (e.g., crosslinked) to a hydrogel forming polymer via the phenylboronic acid or a phenylboronic acid-containing compound. In certain embodiments, the hyaluronic acid is conjugated or linked to a hydrogel forming polymer via a boronic ester bond.

Pyrogallol-containing compounds are known in the art. Pyrogallol, which is also known as 1,2,3-trihydroxybenzene, contains three hydroxyl groups attached to a benzene ring. In certain embodiments, the pyrogallol-containing compound is a polyphenolic compound. In certain embodiments, the pyrogallol-containing compound is a flavonoid. In certain embodiments, the pyrogallol-containing compound is a catechin. Examples of pyrogallol-containing compounds include, without limitation, pyrogallol, gallocathechin, epigallocatechin, epicatechin gallate, epigallocatechin gallate (EGCG), 5-pyrogallol 2-aminoethane (PAE), robinetinidol, 5-hydroxydopamine, tannic acid, gallic acid, 2,3,4-trihydroxybenzaldehyde, 2,3,4-trihydroxybenzoic acid, 3,4,5-trihydroxybenzaldehyde, 3,4,5-trihydroxybenzamide, 5-tert-butylpyrogallol and 5-methylpyrogallol. In certain embodiments, the pyrogallol-containing compound is epigallocatechin gallate (EGCG). EGCG, the most abundant polyphenolic compound in green tea, has various pharmacological properties, including anti-inflammation, antioxidation, antiaging, bactericidal, proangiogenic, and anticancer effects (Shin, et al. (2019) Adv. Funct. Mater., 29 (43): 1903022; Hu, et al. (2018) ACS Nano 12 (4): 3385-3396; Wang, et al. (2019) Food Chem., 271:204-210; Zhao, et al. (2021) Adv. Funct. Mater., 2009442).

The phenylboronic acid-containing compound of the instant invention will generally be phenylboronic acid linked to or substituted with a functional group, optionally via a linker. In certain embodiments, phenylboronic acid is substituted at one or more locations, particularly only one location. In certain embodiments, phenylboronic acid is substituted at the 3, 4, and/or 5 position. In certain embodiments, the phenylboronic acid is substituted with a functional group which can form a bond with the carboxylic acid of hyaluronic acid. In certain embodiments, the phenylboronic acid is substituted with a functional group which can form an amide bond with the carboxylic acid of hyaluronic acid. In certain embodiments, the functional group is —NH₂ or —OH, particularly —NH₂. The functional group may be substituted directly on the phenylboronic acid or may be attached via a linker. In certain embodiments, the linker is an alkyl group, particularly a C₁-C₃ alkyl. In certain embodiments, the phenylboronic acid-containing compound is (3-aminomethylphenyl) boronic acid. Phenylboronic acid-containing dynamic hydrogels have antioxidative properties (Shi, et al. (2020) Carbohydr. Polym., 233:115803; Kong, et al. (2021) Appl. Mater. Today 24:101090).

Hyaluronic acid is a natural glycosaminoglycan polysaccharide with good biocompatibility and biodegradability and has antiadhesive properties (Chandel, et al. (2021) Macromol. Biosci., 21 (3): 2000395; Cai, et al. (2021) Biomacromolecules 22 (12): 4967-4979; Park, et al. (2020) Materials 13 (14): 3056). In certain embodiments, the hyaluronic acid has a molecular weight of at least about 400 kDa, about 500 kDa, about 600 kDa, about 700 kDa, about 800 kDa, about 900 kDa, about 1000 kDa, or about 1100 kDa. In certain embodiments, the hyaluronic acid has a molecular weight of less than about 5000 kDa, about 4000 kDa, about 3000 kDa, about 2000 kDa, about 1800 kDa, about 1600 kDa, about 1400 kDa, about 1300 kDa, about 1200 kDa, about 1100 kDa, or about 1000 kDa. In certain embodiments, the hyaluronic acid has a molecular weight from about 500 kDa to about 1500 kDa, about 600 kDa to about 1200 kDa, about 700 kDa to about 1100 kDa, about 800 kDa to about 1000 kDa, about 850 kDa to about 950 kDa, or about 900 kDa.

In certain embodiments, the hyaluronic acid is conjugated to a zwitterionic compound. Zwitterions are molecules having separate positively and negatively charged groups. In certain embodiments, the zwitterionic compound is conjugated directly to the hyaluronic acid or via a linker. In certain embodiment, the zwitterionic compound is a zwitterionic monomer. Zwitterionic monomers are zwitterionic compounds which can be polymerized to form a polymer. Zwitterionic monomers include a polymerizable group and one or more zwitterionic functional groups, which are a chemical group that includes at least one positively and at least one negatively charged group. Examples of positively charged groups include without limitation: quaternary ammonium groups. Examples of negatively charged groups include without limitation: sulfonate groups, carboxylate groups, phosphonate groups, phosphinate groups, sulfate groups, and —OP(OH)₂O groups, particularly a sulfonate group. In certain embodiments, the positively charged group and negatively charged group are linked via an alkyl group (e.g., a C₁-C₃ alkyl). Examples of zwitterionic functional groups include without limitation: carboxybetaines (which comprise a carboxylate group and a quaternary ammonium group), phosphorylbetaines (which comprise a phosphate group and a quaternary ammonium group), and sulfobetaines (which comprise a sulfonate and a quaternary ammonium group).

Any polymerizable group known in the art can be included in the zwitterionic monomer. In certain embodiments, the polymerizable group comprises a vinylic group. Examples of vinylic groups include without limitation: acryl esters, methacryl esters, acrylamides, and methacrylamides. The polymerizable group may be attached to the zwitterionic functional group via an alkyl (e.g., a C₁-C₃ alkyl).

Examples of zwitterionic monomers include without limitation: N,N-dimethyl-N-(3-methacrylamidopropyl)-N-(3-sulfopropyl) ammonium betaine (SPP); N-(3-sulfopropyl)N-methacryloyloxyethyl-N,N-dimethyl ammonium betaine (SPE); sulphopropyldimethylammonioethyl acrylate, sulphohydroxypropyldimethylammoniopropyl methacrylamide (SHPP), and N,N-dimethyl-N-(2-acryloylethyl)-N-(3-sulfopropyl) ammonium betaine (SPDA; also known as 3-[[2-(acryloyloxy)ethyl](dimethyl) ammonio]-1-propanesulfonate). In certain embodiments, the zwitterionic monomer is N,N-dimethyl-N-(2-acryloylethyl)-N-(3-sulfopropyl) ammonium betaine (SPDA; also known as 3-[[2-(acryloyloxy)ethyl](dimethyl) ammonio]-1-propanesulfonate).

Hydrogel forming polymers are known in the art. Examples of hydrogels include, without limitation, one or more of: gelatin, alginate, chitosan, collagen, silk, fibrin, agarose, chondroitin, elastin, starch, pectin, cellulose, methylcellulose, polyethylene glycol (PEG), polyvinyl alcohol (PVA), sodium polyacrylate, polyacrylamide, starch-acrylonitrile co-polymers, a proteoglycan, elastin, and/or a glycosaminoglycan (e.g., heparin, chondroitin sulfate, or keratan sulfate), other natural or synthetic hydrogels, and derivatives thereof (e.g., del Valle et al., Gels (2017) 3:27). In certain embodiments, the hydrogel is selected from the group consisting of alginate, chitosan, cellulose, agarose, and PVA. In certain embodiments, the hydrogel is biocompatible. In certain embodiments, the hydrogel is biodegradable. In certain embodiments, the hydrogel is non-biodegradable. In certain embodiments, the hydrogel comprises a polymer comprising-OH functional groups or substituents (hydroxyl group). In certain embodiments, the hydrogel polymer comprises PVA. PVA has antiadhesive properties when blended with a synthetic polymer and a natural polymer (Park, et al. (2020) Materials (Basel) 13 (14): 3056).

In certain embodiments, the hydrogel of the instant invention comprises EGCG, PBA, HA, and PVA. In certain embodiments, the hydrogel of the instant invention comprises microgels, comprising HA, EGCG, and PBA, linked/crosslinked with PVA. In certain embodiments, the hydrogel of the instant invention comprises EGCG, PBA, HA, and PVA, wherein EGCG is conjugated to PBA via a boronic ester bond, wherein PBA is conjugated to HA via a linker, wherein PVA is conjugated to PBA via a boronic ester bond, and wherein the HA is optionally conjugated to a zwitterionic monomer via a linker.

Generally, a linker is a chemical moiety comprising a covalent bond or a chain of atoms that covalently attaches two compounds. The linker can be linked to any synthetically feasible position of the compounds, particularly without significantly affecting the activity of the compounds, if applicable. Exemplary linkers may comprise at least one optionally substituted; saturated or unsaturated; linear, branched or cyclic aliphatic group, an alkyl group, or an optionally substituted aryl group. In a particular embodiment, the linker is an optionally substituted alkyl group. The alkyl group may comprise at least one heteroatom (e.g., O, N, or S). In certain embodiments, the alkyl group comprises about 1 to about 10 carbons, about 1 to about 5 carbons, or about 1 to about 3 carbons.

In accordance with the instant invention, methods of inhibiting and/or preventing post-operation adhesions (e.g., post-operative internal tissue adhesions, abdominal adhesions, peritoneal adhesions, recurrent adhesions, etc.) are provided. Examples of internal adhesions include without limitation postsurgical abdominal, nerve, liver, tendon, cardiac, intrauterine, peritendinous, and epidural adhesions. In certain embodiments, the methods comprise applying a hydrogel of the instant invention to at least one surface at the site of the surgery. In certain embodiments, the hydrogel is applied to an injured or damages surface. In certain embodiments, the surgery is abdominal surgery. In certain embodiments, the surgery is adhesiolysis. In certain embodiments, the methods inhibit and/or prevent the recurrence of an adhesion. In certain embodiments, the methods comprise applying a hydrogel of the instant invention to at least one surface of a tissue where adhesions may form. In certain embodiments, the methods comprise applying a hydrogel of the instant invention to at least two sites or tissues between which an adhesion may form. In certain embodiments, the methods comprise applying a hydrogel of the instant invention to one or more of the cecum, abdominal wall, and parietal peritoneal wall. In certain embodiments, the methods comprise applying a hydrogel of the instant invention to the cecum and abdominal wall. In certain embodiments, the hydrogel is applied topically (e.g., applied as a cream). In certain embodiments, the hydrogel is applied by injection. In certain embodiments, the hydrogel is applied by spraying or aerosolization. In certain embodiments, the hydrogel is applied to an implantable device (e.g., hernia mesh) before and/or after implantation into the subject.

In accordance with the instant invention, methods of delivering a compound, drug, and/or therapeutic agent to a subject are provided. The methods comprise administering a hydrogel of the instant invention to a subject, wherein the hydrogel further comprises the compound, drug, and/or therapeutic agent. The method may further comprise adding the compound, drug, and/or therapeutic agent to the hydrogel of the instant invention. In certain embodiments, the compound, drug, and/or therapeutic agent is added to the hydrogel by including the compound, drug, and/or therapeutic agent in a precursor solution of the synthesis of the hydrogel. In certain embodiments, the loaded compound, drug, and/or therapeutic agent can diffuse out sustainably in the target site from the hydrogel. In certain embodiments, the compound, drug, and/or therapeutic agent is released with an accelerated release rate under reactive oxygen species (ROS) environments, such as tumor sites and ischemia areas. In certain embodiments, the hydrogel is administered topically (e.g., applied as a cream). In certain embodiments, the hydrogel is administered by injection. In certain embodiments, the hydrogel is administered by a catheter. In certain embodiments, the hydrogel is administered by spraying or aerosolization.

Therapeutic agents may include, without limitation, drugs, pharmaceuticals, biologics, growth factor, cytokines, chemokines, antibodies, antibody fragments, small molecules, peptides, proteins, nucleic acid molecules, DNA, RNA, and other known biologic substances. In certain embodiments, the therapeutic agent is an antimicrobial (e.g., antibiotic). In certain embodiments, the therapeutic agent is a cytokine (e.g., an anti-inflammatory cytokine). In certain embodiments, the therapeutic agent is an analgesic. In certain embodiments, the therapeutic agent is a chemotherapeutic agent. In certain embodiments, the therapeutic agent is an antioxidant. In certain embodiments, the therapeutic agent is an anti-inflammatory agent. In certain embodiments, the therapeutic agent is an anti-diabetic drug. In certain embodiments, anti-diabetic drugs can be loaded in the hydrogel and the hydrogel injected subcutaneously to a subject in need thereof. Accelerated anti-diabetic drug release can be achieved when the glucose level is high as the hydrogel is glucose responsive.

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antimicrobial. In a particular embodiment, the antimicrobial is a small molecule antibiotic. Examples of antibiotics include, without limitation, beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, ansamycins (e.g., rifamycins, rifampin), aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin).

In accordance with another aspect of the instant invention, methods of treating, inhibiting, and/or preventing a microbial or bacterial infection (e.g., biofilm infection) are provided. In certain embodiments, the method treats, inhibits, and/or prevents an infection associated with an implantable device (e.g., prosthetic or surgical implant). In certain embodiments, the method treats, inhibits, and/or prevents an infection associated with a catheter (e.g., central venous catheter). In certain embodiments, the method treats, inhibits, and/or prevents an infection associated with a stent. In certain embodiments, the hydrogel of the instant invention further comprises an antimicrobial (e.g., antibiotic). In certain embodiments, the methods comprise applying or administering a hydrogel of the instant invention to a subject (e.g., to a site of infection). In certain embodiments, the methods comprise applying a hydrogel of the instant invention to at least one surface of a catheter or implantable device, before and/or after implantation into a subject. In certain embodiments, the hydrogel is applied topically (e.g., applied as a cream). In certain embodiments, the hydrogel is applied by injection. In certain embodiments, the hydrogel is applied by spraying or aerosolization.

In accordance with another aspect of the instant invention, methods of treating, inhibiting, and/or preventing a microbial or bacterial infection (e.g., biofilm infection) on a surface or treating, inhibiting, and/or preventing biofouling (e.g., inhibiting and/or preventing protein absorption and/or microbial growth) are provided. In certain embodiments, the hydrogel of the instant invention further comprises an antimicrobial (e.g., antibiotic). In certain embodiments, the method comprises applying a hydrogel to a stent. In certain embodiments, the method comprises applying a hydrogel to an implantable device (e.g., a temporary or removable implantable device). In certain embodiments, the hydrogels are applied to a catheter (e.g., central venous catheter; e.g., to inhibit and/or prevent catheter biofilm formation). In certain embodiments, the method comprises applying a hydrogel to a stent (e.g., to inhibit and/or prevent stent clotting). In certain embodiments, the hydrogel is used to inhibit and/or prevent.

In certain embodiments, the hydrogel coats an implantable device such as a catheter or stent. The term “coat” refers to a layer of a substance/material on the surface of a structure. Coatings may, but need not, also impregnate the structure. Further, while a coating may cover 100% of the structure, a coating may also cover less than 100% of the surface of the structure (e.g., at least about 75%, at least about 80%, at least about 85%, at least about 90%, at least about 95%, at least about 98%, or more of the surface may be coated).

In accordance with another aspect of the instant invention, methods of treating, inhibiting, and/or preventing inflammation in a subject are provided. In certain embodiments, the hydrogel of the instant invention further comprises an anti-inflammatory. In certain embodiments, the methods comprise applying or administering a hydrogel of the instant invention to a subject (e.g., to a site of inflammation). In certain embodiments, the hydrogel is applied topically (e.g., applied as a cream). In certain embodiments, the hydrogel is applied by injection. In certain embodiments, the hydrogel is applied by spraying or aerosolization.

In accordance with another aspect of the instant invention, methods of treating, inhibiting, and/or preventing cancer in a subject are provided. In certain embodiments, the hydrogel of the instant invention further comprises a chemotherapeutic agent. In certain embodiments, the methods comprise applying or administering a hydrogel of the instant invention to a subject (e.g., to a cancerous site or tumor site). In certain embodiments, the hydrogel is applied topically (e.g., applied as a cream). In certain embodiments, the hydrogel is applied by injection. In certain embodiments, the hydrogel is applied by spraying or aerosolization.

In accordance with another aspect of the instant invention, methods of treating, inhibiting, and/or preventing diabetes in a subject are provided. In certain embodiments, the hydrogel of the instant invention further comprises an anti-diabetic drug. In certain embodiments, the methods comprise applying or administering a hydrogel of the instant invention to a subject. In certain embodiments, the hydrogel is applied topically (e.g., applied as a cream). In certain embodiments, the hydrogel is applied by injection. In certain embodiments, the hydrogel is applied by spraying or aerosolization.

In accordance with another aspect of the instant invention, methods of treating, inhibiting, and/or preventing scar tissue formation and/or promoting wound healing in a subject are provided. In certain embodiments, the hydrogel of the instant invention further comprises an anti-inflammatory. In certain embodiments, the methods comprise applying or administering a hydrogel of the instant invention to a subject (e.g., to the skin, particularly to a wound and/or scar). In certain embodiments, the hydrogel is applied topically (e.g., applied as a cream). In certain embodiments, the hydrogel is applied by injection. In certain embodiments, the hydrogel is applied by spraying or aerosolization.

In accordance with another aspect of the instant invention, methods of moisturizing the skin and/or promoting anti-aging in the skin of a subject are provided. In certain embodiments, the hydrogel of the instant invention further comprises an anti-inflammatory. In certain embodiments, the methods comprise applying or administering a hydrogel of the instant invention to a subject (e.g., to the skin). In certain embodiments, the hydrogel is applied topically (e.g., applied as a cream). In certain embodiments, the hydrogel is applied by injection. In certain embodiments, the hydrogel is applied by spraying or aerosolization.

In accordance with the instant invention, methods of delivering a cell to a subject are provided. The hydrogel is biocompatible and supports the growth and differentiation of encapsulated cells. Cells encapsulated in the hydrogels will be retained longer at the target site and are protected from environmental ROS damage due to the anti-oxidative property of the hydrogel. As such, the survival rate of transplanted cells is increased compared to cells not encapsulated in the hydrogel. The methods comprise administering a hydrogel of the instant invention to a subject, wherein the hydrogel further comprises the cell. The method may further comprise adding the cell to the hydrogel of the instant invention. In certain embodiments, the cell is added to the hydrogel by including the cell in a precursor solution of the synthesis of the hydrogel. In certain embodiments, the hydrogel is administered topically (e.g., applied as a cream). In certain embodiments, the hydrogel is administered by injection. In certain embodiments, the hydrogel is administered by a catheter. In certain embodiments, the hydrogel is administered by spraying or aerosolization. In certain embodiments, the cells are immune cells such as, but not limited to, T cells, B cells, NK cells, macrophages, neutrophils, dendritic cells and modified forms of these cells and various combinations thereof. In certain embodiments, the cells are autologous or allogenic. In certain embodiments, the cells are activated or non-activated. The cells may be cultured in the hydrogel. For example, the cells may be cultured for 1 day, 2 days, 3 days, 4 days, 5 days, or more.

In accordance with another aspect of the instant invention, the hydrogels of the instant invention or precursors thereof are used as a bio-ink for 3D printing. In certain embodiments, the hydrogel is used to print (e.g., via 3D printing) a scaffold (e.g., to support cell growth and/or differentiation). In certain embodiments, the hydrogel is formed using 3D printing and two precursor solutions: 1) a solution comprising hyaluronic acid conjugated to the pyrogallol-containing compound via a phenylboronic acid (e.g., the microgel); and 2) a solution of the hydrogel forming polymer (e.g., PVA). In certain embodiments, the precursors are applied in a 1:1 ratio. Application of the two precursor solution results in the rapid formation of the hydrogel. A compound, drug, and/or therapeutic agent as described herein can be applied to either precursor solution for addition to the hydrogel. In certain embodiments, the hydrogel is printed onto a structure (e.g., an implantable device). In certain embodiments, the hydrogel is printed onto a scaffold. In certain embodiments, the hydrogel is printed and the formed hydrogel is recovered and then used in any of the applications set forth herein (e.g., administered to a subject).

In accordance with another aspect of the instant invention, compositions comprising a hydrogel of the instant invention and at least one carrier (e.g., a pharmaceutically acceptable carrier) are provided. In certain embodiments, composition comprises the hydrogel on an implantable device as described herein (e.g., hernia mesh, catheter, stent, etc.). In certain embodiments, the carrier is water or saline.

In accordance with another aspect of the instant invention, methods of synthesizing the hydrogels of the instant invention are provided. In certain embodiments, the method comprises mixing or combining 1) a composition comprising a microgel comprising a complex comprising hyaluronic acid conjugated to a pyrogallol-containing compound via phenylboronic acid or a phenylboronic acid-containing compound, and 2) a composition comprising a hydrogel forming polymer. In certain embodiments, the compositions are combined using a 3D printer. In certain embodiments, the hydrogel is formed through dynamic covalent bonds between phenylboronic groups of the microgel and hydroxyl groups of the hydrogel forming polymer. In certain embodiments, the ratio of the complex comprising hyaluronic acid conjugated to a pyrogallol-containing compound via phenylboronic acid or a phenylboronic acid-containing compound to the hydrogel forming polymer is about 1:1.

In certain embodiments, the method further comprises synthesizing the microgel comprising a complex comprising hyaluronic acid conjugated to a pyrogallol-containing compound via phenylboronic acid or a phenylboronic acid-containing compound. In certain embodiments, the microgel is synthesized by 1) conjugating the phenylboronic acid-containing compound to the phenolic hydroxyl groups of the pyrogallol-containing compound, and 2) conjugating a functional group (e.g., amino group) of the phenylboronic acid-containing compound to the carboxyl groups of hyaluronic acid. In certain embodiments, the molar ratio of the phenylboronic acid-containing compound to the pyrogallol-containing compound is about 8 to about 1. In certain embodiments, the conjugation ratio of the pyrogallol-containing compound relative to the phenylboronic acid-containing compound is about 0.5 to about 1. In certain embodiments, the phenylboronic acid-containing compound substitution is about 10% to about 100%. In certain embodiments, the size of microgel is tunable from about 200 nm to about 100 μm.

The hydrogels of the instant invention (optionally in a composition) can be administered to an animal, in particular a mammal, more particularly a human. The pharmaceutical compositions of the instant invention may also comprise at least one other compound or therapeutic agent such as an antiviral agent or antimicrobial. The additional compound may also be administered in a separate pharmaceutical composition from the compositions of the instant invention. The pharmaceutical compositions may be administered at the same time or at different times (e.g., sequentially).

The dosage ranges for the administration of the compositions of the invention are those large enough to produce the desired effect (e.g., curing, relieving, treating, and/or preventing the disease or disorder, the symptoms of it, or the predisposition towards it). The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The hydrogels described herein will generally be administered to a patient as a pharmaceutical composition. The term “patient” as used herein refers to human or animal subjects. These compositions may be employed therapeutically, under the guidance of a physician.

The pharmaceutical compositions of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the components in the chosen medium may be varied and the medium may be chosen based on the desired route of administration of the pharmaceutical composition. Except insofar as any conventional media or agent is incompatible with the component to be administered, its use in the pharmaceutical composition is contemplated.

The dose and dosage regimen of compositions according to the invention that are suitable for administration to a particular patient may be determined by a physician considering the patient's age, sex, weight, general medical condition, and the specific condition for which the components are being administered and the severity thereof. The physician may also take into account the route of administration, the pharmaceutical carrier, and the component's biological activity.

Selection of a suitable pharmaceutical composition will also depend upon the mode of administration chosen. For example, the components of the invention may be administered by direct injection. In this instance, a pharmaceutical composition comprises the component dispersed in a medium that is compatible with the site of injection.

Hydrogels of the instant invention may be administered by any method. For example, the components of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the hydrogel is administered by injection. In a particular embodiment, the hydrogel is administered as a spray. In a particular embodiment, the hydrogel is administered by aerosolization or by an aerosol spray. In a particular embodiment, the hydrogel is administered topically. Pharmaceutical compositions for injection are known in the art. If injection is selected as a method for administering the hydrogel, steps should be taken to ensure that sufficient amounts of the hydrogel and its components reach their target cells to exert a biological effect. Dosage forms for topical administration include, without limitation, creams, gels, ointments, salves, patches and transdermal delivery systems.

Pharmaceutical compositions containing a hydrogel of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., topical or direct injection.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of a hydrogel may be determined by evaluating their toxicity in animal models. Various concentrations of components in pharmaceutical composition may be administered to mice, and the minimal and maximal dosages may be determined based on the beneficial results and side effects observed as a result of the treatment. Appropriate dosage unit may also be determined by assessing the efficacy of the treatment in combination with other standard drugs. The dosage units may be determined individually or in combination with each treatment according to the effect detected.

The pharmaceutical composition comprising the hydrogel may be administered at appropriate intervals until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, treat, or lessen the symptoms of a particular disorder or disease. For example, the treatment of an infection herein may refer to reducing, curing, and/or relieving the infection, the symptom(s) of it, or the predisposition towards it.

As used herein, the term “small molecule” refers to a substance or compound that has a relatively low molecular weight (e.g., less than 4,000, or less than 2,000 Da). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains).

The term “alkyl,” as employed herein, includes straight or branched chain hydrocarbons containing 1 to about 30 carbons in the normal/main chain. The hydrocarbon chain of the alkyl groups may be interrupted with one or more heteroatom (e.g., oxygen, nitrogen, or sulfur). An alkyl may, optionally, be substituted (e.g. with fewer than about 8, fewer than about 6, or 1 to about 4 substituents). The term “lower alkyl” refers to an alkyl which contains 1 to 3 carbons in the hydrocarbon chain. Alkyl substituents include, without limitation, alkyl (e.g., lower alkyl), alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, ether, ester, thioester, nitrile, nitro, amide, carbonyl, carboxylate and thiol.

The following examples illustrate certain embodiments of the invention. They are not intended to limit the invention in any way.

### Example 1

### Materials and Methods

### Preparation of PBA-EGCG Complex

The phenylboronic acid (PBA)-epigallocatechin-3-gallate (EGCG) complex was prepared through the formation of two reversible boronic ester bonds between the catechol groups in EGCG and PBA. Briefly, 62.1 mg of PBA (particularly (3-aminomethylphenyl) boronic acid hydrochloride; Sigma-Aldrich, St. Louis, MO) were dissolved in 300 μL of DMSO at a concentration of 207 mg/mL (1 M), and 18.9 mg of EGCG (95%, Asta Tech, Bristol, PA) were added into 300 μL of DMSO at a concentration of 63 mg/mL (0.125 M). After adding the EGCG solution into the PBA solution, the mixture was mixed by a vortex mixer for 30 seconds and was left to rest for 15 minutes at room temperature to drive complex formation. The ultimate molar ratio of PBA and EGCG was 8:1. The EGCG molar ratio relative to PBA was 12.5:100. Chemical structures are provided below.

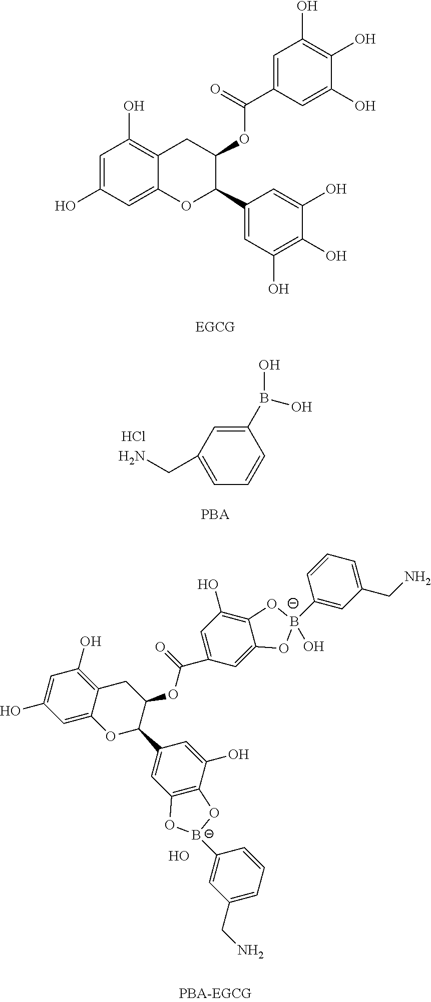

### Synthesis of HA-PBA-EGCG (HPE) Microgels

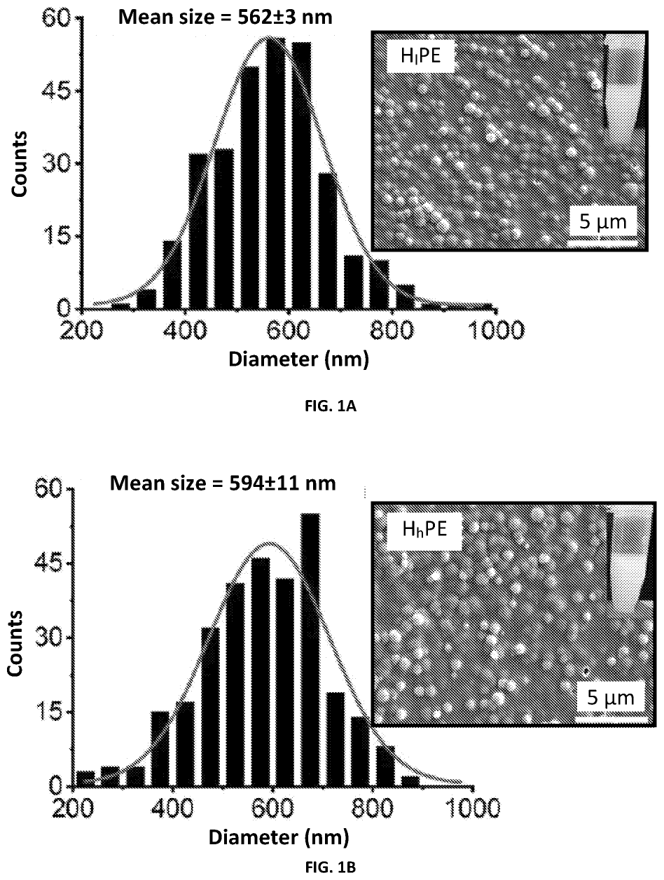

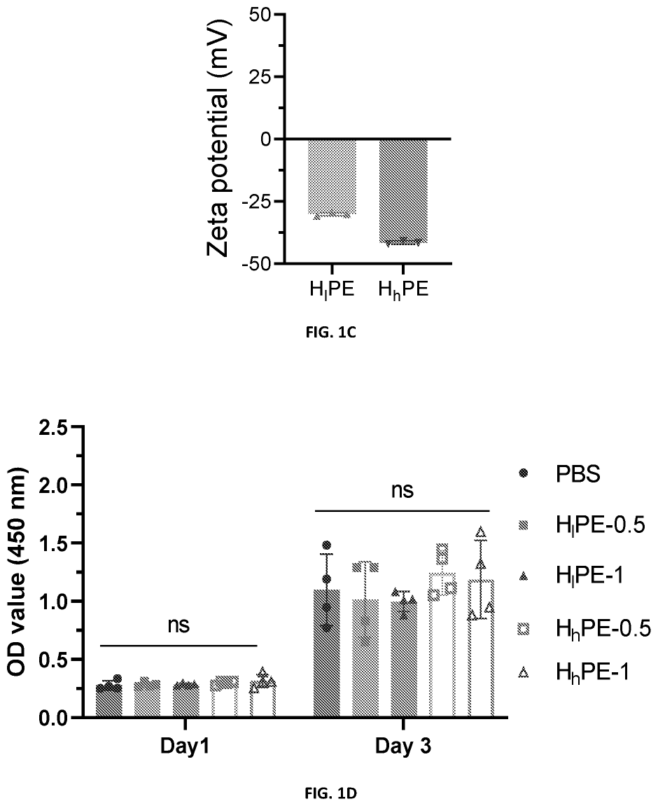

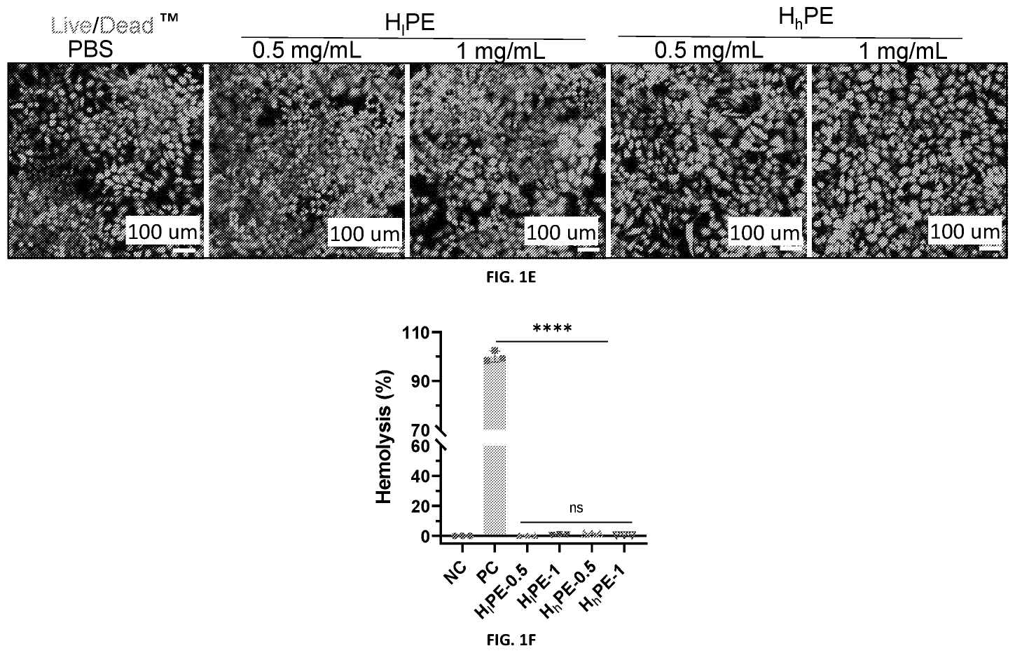

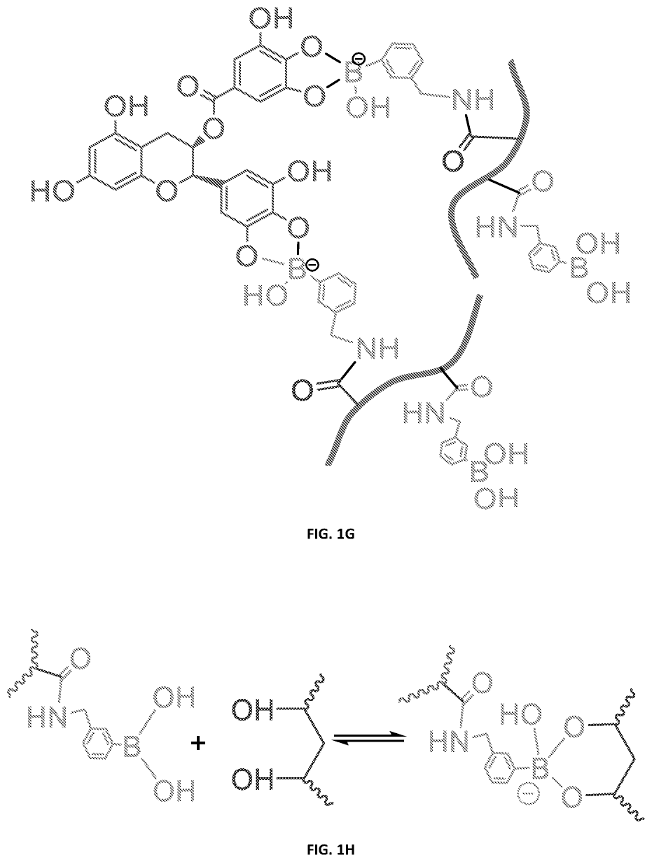

The hyaluronic acid (HA)-PBA-EGCG microgel (see FIG. 1G) was prepared by conjugating the PBA-EGCG complex to HA (40 kDa for HₗPE, 600-1200 kDa for HₕPE; Bloomage Biotech, Jinan, China), using 4-(4,6-dimetnoxy-1,3,5-triain-2-yl)-4-methylmorpholinium chloride (DMTMM) (TCI, Portland, OR) as coupling agent. Typically, 125 mg of HA were dissolved in 15 mL of de-ionized water under stirring. Next, the prepared PBA-EGCG complex and DMSO solution (600 μL) and DMTMM (182.2 mg) were added to the HA solution separately. After all the agents were dissolved, the pH of the solution was adjusted to 6.5 using a 2-(N-morpholino) ethanesulfonic acid (MES) buffer (1 M; Fisher Chemical, Hampton, NH). Then the reaction mixtures were stirred at room temperature for 3 days. The final mixtures were purified via dialysis (molecular weight cutoff of 10 kDa, MWCO, Spectrum™) against deionized water for 3 days at room temperature to remove DMSO, with the water changed twice every day. The dialyzed solution was lyophilized using a benchtop lyophilizer (Labconco; Kansas City, MO) to obtain the HPE microgels. The HPE microgels were stored in the dark before use.

### Characterization of HPE Microgels

The chemical structures of the HPE microgels were determined by nuclear magnetic resonance (¹H NMR) and FTIR. The ¹H NMR spectra were recorded on a Brucker 500 MHz spectrometer, using D₂O and d6-DMSO as the solvents. The FT-IR spectra were recorded using an FTIR spectrometer (Nicolet™ iS50) in the range 500-4000 cm⁻¹. The zeta potentials of the HPE microgels were measured at 25° C. by using dynamical light scattering (DLS, Zetasizer Nano ZS90, Malvern Instruments, Malvern, UK). The HPE microgels were quickly immersed into liquid nitrogen and lyophilized for 24 hours. Subsequently, the samples were sputter-coated with a thin layer of gold in a vacuum chamber. The microstructures of the samples were visually characterized by an scanning electron microscope (SEM) (FEI Quanta™ 200). The size of the HPE microgels was calculated using ImageJ software.

### Synthesis of HPE-PVA Hydrogels

A schematic of the linkage of the HPE to PVA is provided in FIG. 1H. The lyophilized HPE microgels were resuspended in deionized water (2% w/v), and polyvinyl alcohol (PVA) (13-23 kDa, Sigma) was dissolved in deionized water (2% w/v). Specifically, the samples were: HₗPE-PVA: HA MW=40 kDa, HPE (w/w): 1%, PVA (w/w): 1%; and HₕPE-PVA: HA MW=900 kDa, HPE (w/w): 1%, PVA (w/w): 1%. The HPE-PVA hydrogels formed rapidly (about 10 seconds) after both solutions were mixed at a 1:1 volume ratio. For the preparation of fluorescein labeled HPE-PVA hydrogels, cyanine 7.5 (Cy7.5)-labeled HA was first synthesized by an amidation reaction between fluorescein cyanine 7.5 amine (Lumiprobe, Cockeysville, MD; 260C0, 0.1% w/w relative to HA) and HA. Subsequently, the Cy7.5-labeled HPE and Cy7.5-labeled HPE-PVA hydrogels were prepared following the described procedure described.

### Characterization of HPE-PVA Hydrogels

The rheological analysis of the cream hydrogels was conducted at 37° C. using an AR1500ex rheometer (TA Instruments, New Castle, DE). Rheological tests were performed with a 20 mm diameter parallel-plate geometry. Briefly, a 2 wt % HPE solution and 2 wt % PVA solution were prepared for the rheological test. The time-sweep method was performed under a strain of 1% for 5 minutes at 37° C. to evaluate the stability of the hydrogels. Frequency-sweep tests from 0.1 to 100 rad/s were conducted under 1% strain at 37° C. to evaluate the storage modulus (G′) and loss modulus (G″) of the hydrogels. Step-strain measurements were performed with the shear strain ranging from 0.1% to 300% at a frequency of 1 Hz. Steady shear viscosity rheology of HPE-PVA was also performed with a shear rate varying from 0.1 to 100 s⁻¹. The samples were run in triplicate.

The injectability of the hydrogel was also tested macroscopically. First, for clear observation, the HPE solution was mixed with either a red food color dye or a blue food color dye. After gelation, the HPEPVA hydrogels were loaded into an insulin syringe capped with a 29-gauge needle to evaluate whether the hydrogel could be extruded through the needle after manually pushing the syringe plunger. To evaluate the self-healing ability, the two separate parts of the HPEPVA hydrogel (with red or blue dyes, respectively) were brought into contact with each other in a plastic mold. After 50 seconds, the healed hydrogel was held up and both ends were pulled on with tweezers for observation.

### In Vitro Degradation of HPE-PVA Hydrogels

In vitro degradation of the HPE-PVA hydrogel was monitored by measuring its weight loss over time. 100 μL of the hydrogels was lyophilized and the initial weight was recorded. Then, 100 μL of the hydrogel was immersed in 1 mL of PBS and incubated in a shaking bath at 37° C. at 125 rpm. At each predetermined time point, HPE-PVA samples were freeze-dried and then weighed. The remaining weight (RW) was calculated with the equation: RW (%)=Wᵣ/Wᵢ×100%, where Wᵢ and Wᵣ are the dry weight of the initial hydrogels and the remaining weight of the frozen dried hydrogels after degradation at different time points, respectively. The degradation media were refreshed daily.

### In Vitro Cytocompatibility and Anti-Cell Adhesion

The cytocompatibilities of HPE (HₗPE and HₕPE) and HPE-PVA (HₗPE-PVA and HₕPE-PVA) hydrogels were evaluated by two methods, i.e., Live/Dead™ imaging and a cell counting kit-8 (Abcam, Cambridge, UK; ab228554, CCK8) assay. L929 fibroblasts (ATCC, Manassas, VA) were seeded into a 48-well plate at a density of 5×10³ cells per well and cultured overnight in DMEM (Gibco) culture medium containing 10% (v/v) fetal bovine serum (FBS, Gibco™) and 1% (v/v) penicillin-streptomycin (P/S, Gibco™). Then, the medium was replaced with fresh medium containing HPE microgels (0.5 or 1 mg/mL) or 100 μL of HPE-PVA hydrogels. The samples were further cultured at 37° C. for 3 days. After staining them with 500 μL of calcein-AM/propidium iodide dyes (Invitrogen, Waltham, MA) for 30 minutes, the cells were observed under a confocal laser scanning microscope (CLSM, LSM710) to show the green (live cells) and red (dead cells) fluorescence. The proliferation of L929 cells in the presence of HPE microgels and HPE-PVA hydrogels was evaluated by using the previously described culture method and the CCK8 assay (Abcam). After culturing for 24 and 72 hours, the medium was discarded, and the cells were washed with PBS. About 500 μL of complete medium, containing 50 μL of CCK8 solution, was added to each well, followed by incubation for 4 hours. The optical density at 460 nm (OD460) of each well was measured using a microplate reader (BioTek Synergy™ H1 model). The untreated cells incubated with the normal culture medium served as a control.

The anti-cell adhesion was evaluated by using L929 cells as previously reported, with slight modification (Zhang, et al. (2021) Adv. Funct Mater., 31 (10): 2009431; Yu, et al. (2021) Adv. Mater., 33 (16): 2008395). Briefly, the HPE-PVA hydrogels were placed in a 48-well plate and incubated at 37° C. for 30 minutes. L929 cells were seeded onto the surface of the hydrogels at a density of 1×10+ cells per well and then incubated for 18 hours at 37° C. After discarding the medium, the hydrogels were gently rinsed with DPBS to remove the unattached cells. L929 cells attached to the surface were stained by a Live/Dead™ assay, and the morphology was observed by a CLSM. L929 cells seeded on the tissue culture plate were used as a control.

### In Vitro Hemolysis Assay

Human red blood cells (RBCs) were provided by the Elutriation Core Facility at the University of Nebraska Medical Center (UNMC). The hemolytic study was carried out by using HPE microgels and HPE-PVA hydrogels according to a modified protocol (Yu, et al. (2021) Adv. Mater., 33 (16): 2008395). Briefly, after being washed 3 times, RBCs were resuspended in PBS that contained HPE microgels (0.5 or 1 mg/mL) or HPE-PVA hydrogels (100 μL) and then incubated at 37° C. for 3 hours. Then, the RBC suspensions were centrifuged at 2000 rpm for 10 minutes. The supernatant of the RBC suspension was photographed, and the OD values were measured at 541 nm. The RBCs in PBS only were used as a negative control (NC), while RBCs in a 1% Triton™ X-100 (Sigma) aqueous solution served as a positive control (PC). The hemolysis ratio was calculated according to the following equation: Hemolysis (%)=(ODₛₐₘₚₗₑₛ−OD_{NC})/(OD_{PC}−OD_{NC})×100.

### In Vitro Antioxidative Effects of HPE-PVA Hydrogels

The antioxidative efficiency of HPE-PVA (HₗPE-PVA and HₕPE-PVA) hydrogels was evaluated by two methods, i.e., an ROS detection assay and a free radical scavenging assay. First, an H2DCFDA assay (EMD Millipore, Burlington, MA) was used to detect and qualify the intracellular ROS in response to hydrogen peroxide (H₂O₂). Briefly, the extract medium of HPE-PVA hydrogels (10 mg/mL, 20 μL/well) was first prepared after being incubated in 200 μL of cell culture medium overnight at 37° C. The L929 cells were seeded in 96-well culture plates with 1×10⁴ cells per well and incubated for 24 hours. Subsequently, the cells were treated with 200 μL of collected hydrogel extract medium containing H₂O₂ (800 μM). After incubation at 37° C. for 1 hour, the cells were washed with DPBS three times and treated with culture medium containing 10 μM H2DCFDA for another 20 minutes in the dark at 37° C. Then, the intracellular ROS concentration was measured by detecting the DCF fluorescence intensity (Eₓ₄₈₈/Eₘ₅₂₅) through a microplate reader. The cells treated with culture medium containing PBS only and H₂O₂ only served as negative and positive controls, respectively. The cells were also imaged using a CLSM to visualize the DCF fluorescence. The radical scavenging activity of HPE-PVA hydrogels was measured by observing colorimetric changes of DPPH (Alfa Aesar) according to the previous report (Zhao, et al. (2021) Adv. Funct. Mater., 31:2009442). Briefly, the hydrogels (100 μL) and 100 μM DPPH were dispersed in 3 mL of ethanol. The mixture was stirred and incubated in a dark place at 37° C. for half an hour. Next, the absorption (A) of the samples was tested at 517 nm using a UV-vis instrument. The DPPH free radical scavenging efficiency was calculated by the following formula: DPPH radical scavenging %=(A₀−Aₕ)/A₀×100, where A₀ and Aₕ are the absorptions of the blank (DPPH+ethanol) and the absorption of the hydrogel sample (DPPH+ethanol+HPE-PVA hydrogel), respectively.

### In Vitro Anti-Inflammatory Activity of HPE-PVA Hydrogels

The anti-inflammatory activity of HPE-PVA hydrogels was evaluated using human monocyte derived macrophages similarly to reported methods (Liu, et al. (2022) Bioact. Mater., 14:61-75). Monocytes were supplied by the Elutriation Core Facility at UNMC. About 5×10⁵ monocytes were seeded on sterile glass coverslips in 24-well plates and then cultured for 6 days in RPMI 1640 medium (Gibco) containing 10% FBS, 1% P/S, and 50 ng/mL recombinant human granulocyte macrophage colony-stimulating factor (GM-CSF, PeproTech, Cranbury, NJ, 300-03). After 6 days, the cell culture medium was changed to fresh medium containing HPE-PVA hydrogels and GM-CSF and cultured overnight. Then, the macrophages were stimulated with 100 ng/ml lipopolysaccharide (LPS, PeproTech) and 20 ng/mL animal-free recombinant human interferon-gamma (IFN-γ, PeproTech, AF-300-02) in the presence of HPE-PVA hydrogels. The experiment was divided into four groups: the negative control group (without stimulation), the positive control group (with LPS/IFNγ stimulation), and treated groups (HₗPE-PVA and HₕPE-PVA with LPS/IFNγ). After overnight incubation, the culture medium was collected to detect the cytokine levels of IL-1β and TNF-α by using enzyme-linked immunosorbent assay (ELISA) kits (Bio-Techne, Minneapolis, MN). qPCR was also performed to evaluate the gene expressions of NFκB and TNF-α.

### In Vivo Antiadhesion Evaluation in a Mouse Cecum-Abdominal Wall Defect Model

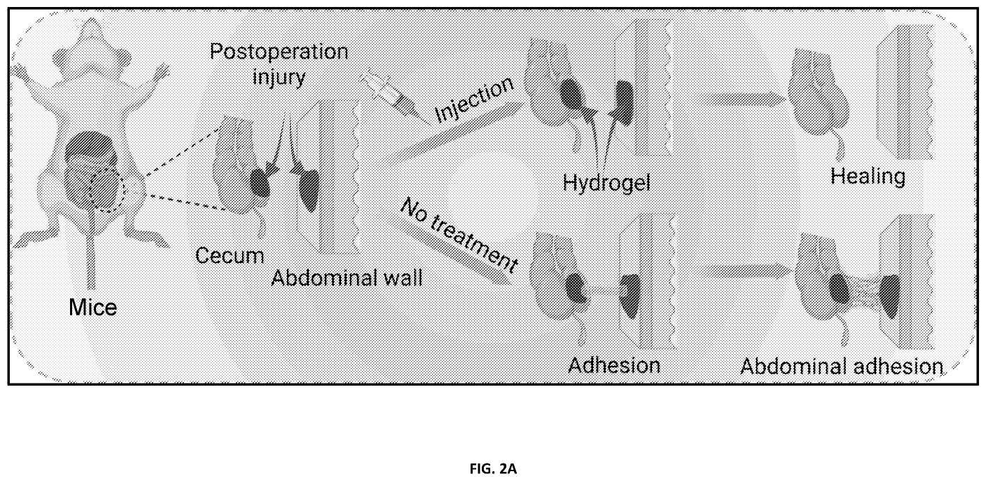

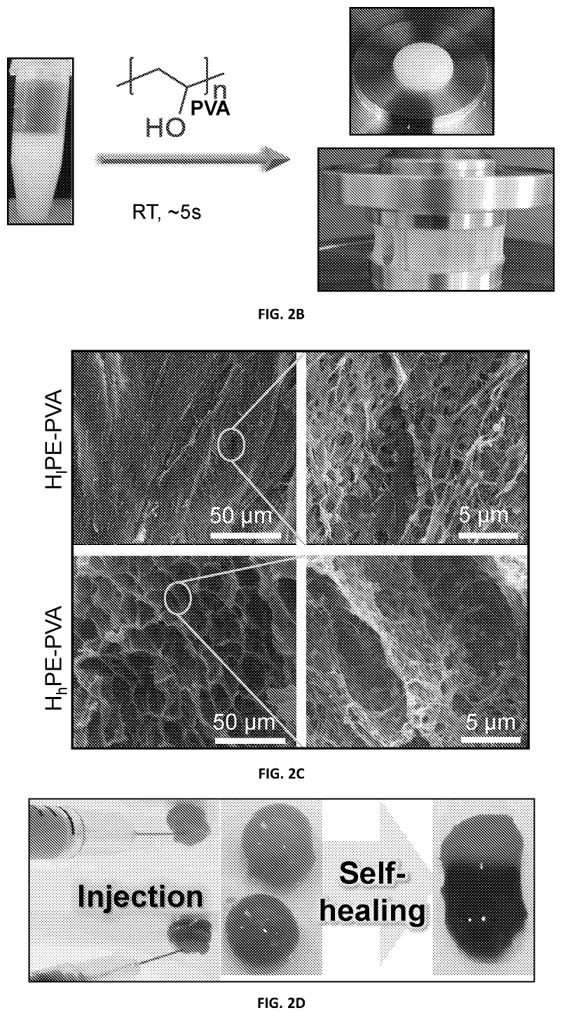

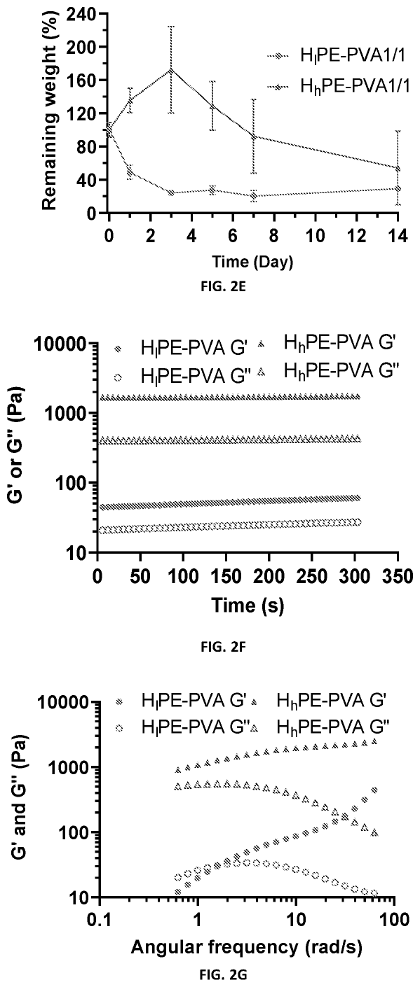

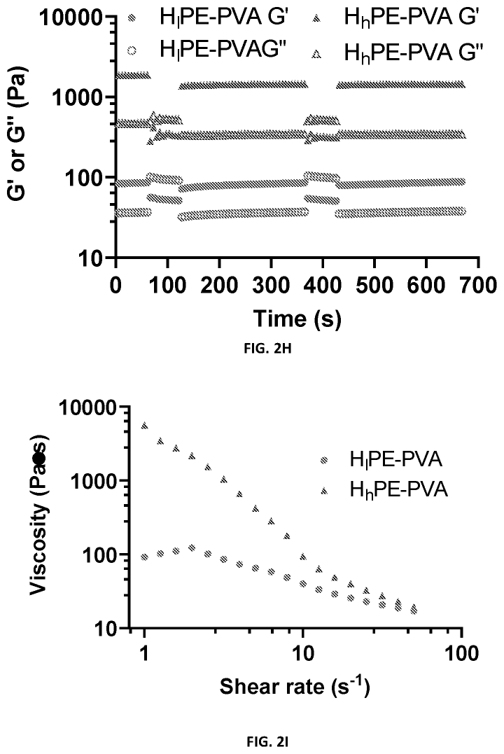

The in vivo experimental design is illustrated in FIG. 2A. Briefly, the in vivo experimental design comprises injuries on the cecum and the abdominal wall created by abrasion and excision, respectively, followed by the covering of both traumatized surfaces with the hydrogels. C57BL/6 mice (female, 12 weeks old, weighing 20±5 g, Charles River Laboratories, Wilmington, MA) were acclimatized for 1 week and then used for the mouse cecum-abdominal wall adhesion model. All mice were randomly divided into five groups, including Sham, Injury-only, HₗPE-PVA hydrogel, HₕPE-PVA hydrogel, and Seprafilm® groups, with eight mice in each group. A sidewall defect-cecum abrasion was performed according to protocol (Zindel, et al. (2021) Science 371 (6533): 993; De Clercq, et al. (2016) Biomaterials 96:33-46). Briefly, the mice were anesthetized using isoflurane via a small animal gas anesthesia machine, and the abdominal hairs were shaved. The peritoneum was opened by a 2 cm long incision along the linea alba on the abdominal wall to expose the cecum. Subsequently, the surface injury of the cecum was induced by gently abrading the cecum with sterile surgical gauze (˜100 times) until a petechial hemorrhage was observed on the surface of the cecum. Next, the abdominal wall was scraped with a scalpel to form a peritoneal injury with an area of 1×1 cm² without hemostasis. The abraded cecum was placed opposite to the abdominal wall defect. The mice in the Injury-only group were washed with 50 μL of PBS on both the injured cecum and injured abdomen, and the mice in both the HₗPE-PVA and HₕPE-PVA hydrogel groups were treated with 50 μL of the HlPE-PVA and HhPE-PVA hydrogels, respectively, on the injury sites, including injured cecum and abdomen. The mice in the Seprafilm® group were treated with a Seprafilm® film (1×1 cm², Baxter Advanced Surgery) on the injury sites between the cecum and injured abdominal wall. Finally, the abdominal wall and skin were sutured with 5-0 and 6-0 sutures layer by layer. All surgical procedures were performed under sterile conditions.

### Gross Observation and Adhesion Score Evaluation

The mice were humanely euthanized with CO₂ asphyxiation and cervical dislocation, following Animal Welfare Act guidelines, at POD 14. After skin dissection, the sutured line was reopened to expose the injured/repaired site to evaluate the results of the adhesion formation and injured tissue healing. The degree of adhesions in the mice was evaluated by a double-blind scoring method, according to the standard adhesion scoring system (De Clercq, et al. (2016) Biomaterials 96:33-46; Yu, et al. (2021) Adv. Mater., 33 (16): 2008395): score 0: no adhesion; score 1: one thin filmy adhesion that requires gentle blunt dissection to be divided; score 2: more than one thin adhesion; score 3: one thick adhesion with a focal point; score 4: one thick adhesion with plantar attachment or more than one thick adhesion with focal points; score 5: very thick vascularized adhesions or more than one plantar adhesion.

### Histological, IF, and IHC Staining

Histopathological examination was performed to observe the pathological changes of the cecum and abdominal wall after surgery. At POD 14, the cecum and abdominal wall or adhesion tissue of each group were harvested. The harvested specimens were fixed with 4% paraformaldehyde (PFA) solution, dehydrated using ethanol, and embedded into paraffin. Then, the paraffin-embedded specimens were sectioned into 5 μm thick sections. Subsequently, the sections were stained with H&E and Masson's trichrome staining. The images were examined blinded. Additionally, the main organs (heart, liver, spleen, lung, and kidney) were histologically examined to evaluate the in vivo biocompatibility. For IF staining, the isolated specimens (adhesion tissue, cecum, and abdominal wall) were dissected and then fixed in 4% PFA solution and dehydrated in 30% sucrose. Then, the specimen was embedded in OCT within a cryo-mold and cut into 12 μm-thick sections at −20° C. using a cryotome (CM1850, Leica, Teaneck, NJ). After being immerged in methanol for 5 minutes, all sections were permeabilized and blocked with 0.1% Triton™ X-100 and 5% goat serum in PBS for 2 hours, followed by incubation with primary antibodies overnight at 4° C. In detail, primary antibodies, including CD68 (AbboMax, San Jose, CA; 500-6074, 1:100), iNOS (Novus Biologicals, Littleton, CO; NB300-605, 1:50), Serpin E1/PAI-1 (Novus Biologicals, NBP1-19773, 1:100), and tPA (Invitrogen, 10147-1-AP, 1:50), were used in this study. After sequential incubation with the appropriate secondary antibodies for 2 hours and DAPI (Invitrogen, D1306, 1:1000) for 30 minutes at room temperature, the stained samples were imaged using a CLSM. The inflammatory effect was semi-quantitatively evaluated by comparing iNOS and CD68 positive areas, which were measured using ImageJ software.

For the measurement of intracellular ROS in vivo, DHE (Biotium, Fremont, CA; 10057) staining was performed. Briefly, the fresh specimen sections from different groups were incubated with DHE (5 mM) for 20 minutes and DAPI at 37° C. in the dark.

The generated ethidium fluorescence was imaged using a CLSM. The ROS intensity was semi-quantitatively calculated by using ImageJ software as follows: the DNE/DAPI ratio (positive area %)=the positive area of DHE fluorescence/the positive area of DAPI fluorescence×100.

For abdominal adhesion immunohistochemistry, sections were deparaffinized with CitriSolv™ (Decon Laboratories, Inc., King of Prussia, PA) and rehydrated with graded ethanol. For antigen retrieval, the sections were treated with 10 mM sodium citrate buffer pH 6.0 (Sigma) in a 75° C. water bath for 20 minutes. Next, the sections were preincubated with 3% hydrogen peroxide in methanol for 15 minutes to deactivate the endogenous peroxidase and then blocked with blocking serum for 1 hour at room temperature. The sections were then incubated with a rabbit recombinant multiclonal anti-S100A8+S100A9 primary antibody for about 20 minutes (Abcam, RM1038, 1:5000). After being washed with PBS, the sections labeled with the anti-S100A8+S100A9 primary antibody were incubated with a biotinylated goat antirabbit IgG (Vector Laboratories) for 1 hour at room temperature. After washing with PBS, an avidin-biotin complex conjugated to peroxidase (Vector Laboratories, Newark, CA) was applied for 30 minutes. The bound complexes were visualized by adding a DAB substrate (3,3′-diaminobenzidine, Vector Laboratories) to the sections for 10 minutes. The images of the sections were taken by a light microscope. The sections were stained with a rabbit recombinant anti α-SMA (Cell Signaling, Danvers, MA; D4K9N, 1:300) by following similar procedures.

qPCR Analysis

The total RNA from the human macrophages or mouse specimens was analyzed by qPCR. Briefly, the total RNA was isolated from cells or freshly collected tissues using QIA-Shredder® and RNeasy® mini-kits (QIAGEN, Hilden, Germany). Complementary DNA (cDNA) was synthesized by following the protocol of an iScript™ cDNA synthesis kit (BioRad Laboratories, Hercules, CA). For the quantitative analysis of the total RNA expression, qPCR amplification was performed in a StepOnePlus™ Real-Time PCR System (Thermo Scientific) with the use of SYBR® Green Supermix (Bio-Rad). The cDNA samples were analyzed for genes of interest and for the housekeeping gene 18S (for human macrophages) and GAPDH (for mouse tissues). The target genes included TNF-α and NFκB for human cells and iNOS, TGF-β1, and IL-6 for mouse tissues, as listed in Table 2. The relative transcription level of each target gene was calculated using the comparative Ct (2^{-ΔΔCt}) method.

TABLE 2
List of primers used for real-time qPCR.

| | Gene | | |
|---|---|---|---|
| Species | name | Forward primer | Reverse primer |
| Human | 18s | CTTAGTTGGTGGAGCGATTT | GCTGAACGCCACTTGTCC |
| | | (SEQ ID NO: 1) | (SEQ ID NO: 2) |
| | NFκB | GCAGCACTACTTCTTGACCACC | TCTGCTCCTGAGGATTGACGTC |
| | | (SEQ ID NO: 3) | (SEQ ID NO: 4) |
| | TNF-α | CTCTTCTGCCTGCTGCACTTG | ATGGGCTACAGGCTTGTCACTC |
| | | (SEQ ID NO: 5) | (SEQ ID NO: 6) |
| Mouse | GAPDH | CATCACTGCCACCCAGAAGACTG | ATGCCAGTGAGCTTCCCGTTCAG |
| | | (SEQ ID NO: 7) | (SEQ ID NO: 8) |
| | iNOS | TTGGTGAAGGGACTGAGCTGTT | ACTTCCAGGGGCAAGCCAT |
| | | (SEQ ID NO: 9) | (SEQ ID NO: 10) |
| | TGF-β1 | CGCCTGAGTGGCTGTCTTTTG | TGGGGCTGATCCCGTTGAT |
| | | (SEQ ID NO: 11) | (SEQ ID NO: 12) |
| | TNF-α | GAGTGACAAGCCTGTAGCCCA | TTGAGATCCATGCCGTTGGC |
| | | (SEQ ID NO: 13) | (SEQ ID NO: 14) |
| | IL-6 | AGACAAAGCCAGAGTCCTTCAGA | AGGAGAGCATTGGAAATTGGGG |
| | | (SEQ ID NO: 15) | (SEQ ID NO: 16) |

### Western Blotting Analysis

The protein levels were analyzed using the Western blotting method according to protocol. Briefly, the harvested specimens were homogenized with a lysing buffer (10 mM Tris, 1 mM EDTA, 150 mM NaCl, 1% SDS, 1 mM PMSF; pH 7.4) plus a protease inhibitor cocktail (100 μL/mL, P2714, Sigma-Aldrich). After centrifugation at 12,000 g for 20 minutes at 4° C., a bicinchoninic acid protein assay kit (Thermo Fisher Scientific) was used to determine the total protein concentration in the supernatant. After mixing each sample with a loading buffer and heating it for 5 minutes at 100° C., an equal amount of protein for each sample (40 ug/well) was loaded in the stacking gel and separated on a 12% sodium dodecyl sulfate (SDS)-polyacrylamide running gel. The proteins were electrophoretically transferred to a PVDF membrane (EMD Millipore) at 100 V for 1.5 hours. The membrane was blocked with 5% nonfat milk for 1 hour and incubated overnight at 4° C. with rat anti-TNFα antibody (Invitrogen, MM350C, 1:1000), mouse anti-IL-1β antibody (Invitrogen, MM425B, 1:1000), rabbit anti-tPA antibody (Invitrogen, 10147-1-AP, 1:1000), rabbit anti-Serpin E1/PAI-1 antibody (Novus Biologicals, NBP1-19773, 1:1000), rabbit recombinant multiclonal anti-S100A8+S100A9 antibody (Abcam, RM1038, 1:1000), and mouse anti-HSP70 antibody (Santa Cruz, sc-7298, 1:5000). After washing with PBST, the membrane was incubated for 1 hour with peroxidase-conjugated goat antirat IgG-800 (926-Li-Cor Bioscience, Lincoln, NE; 32219, 1:5000), goat antimouse IG-680 (Cell Signaling, 5470, 1:5000), and goat antirabbit IG-800 (Cell Signaling, 5151, 1:5000). The protein signal was detected by Odyssey® Imager (Li-Cor).

### Proteomics Analysis

The fresh specimens were collected as described above and homogenized with a pH 7.4 lysing buffer (25 mM Tris-HCl, 150 mM NaCl, 10×EDTA, a 10 μL/mL protease inhibitor cocktail, 1% SDS) using sonification in an ice bath (on 3 seconds, off 2 seconds, 3 times). After centrifugation at 14,000 rpm for 10 minutes at 4° C., the protein concentration in the supernatant was measured by a MicroBCA™ kit (Thermo Fisher Scientific). Finally, the supernatant was stored at −80° C. before use. For quantitative proteomics, the details are available in the Supporting Information.

### Statistical Analysis

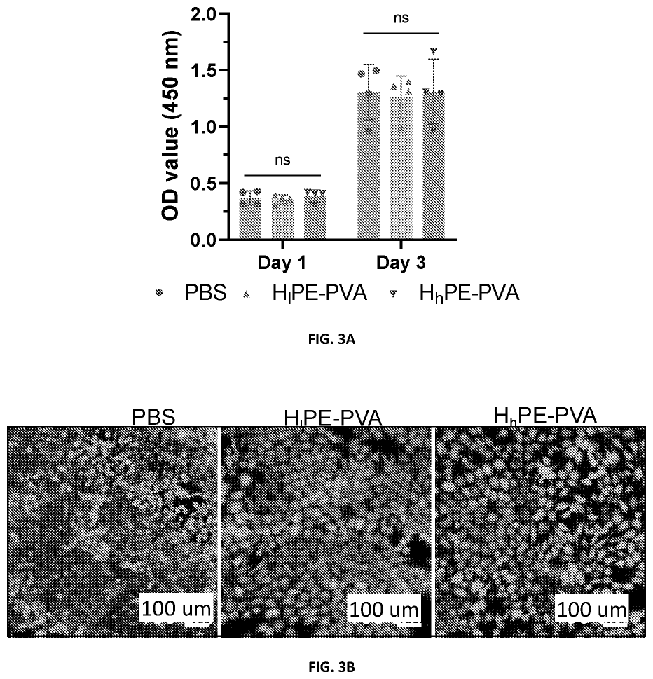

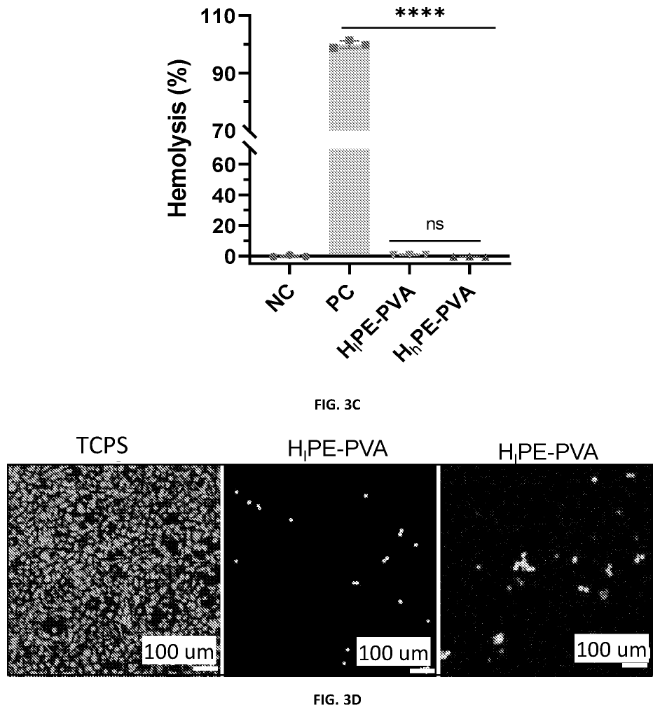

All quantitative data were expressed as mean±standard deviation (SD). FIGS. 2D and 3A were determined by two-way analysis of variance (ANOVA) with a Tukey's multiple comparison test using GraphPad Prism software. All other statistical analyses were performed by one-way ANOVA with a Tukey's post hoc test. A p-value of <0.05 was considered significant for all statistical analyses.

### In Vivo Retention Study

The retention of the HPV-PVA hydrogels in the peritoneal cavity was evaluated by performing IVIS (IVIS Spectrum, PerkinElmer), which detects the fluorescence from Cy7.5-labeled HPE-PVA hydrogels. The Cy7.5-labeled HₗPE-PVA and HₕPE-PVA hydrogels were applied to the sidewall defect and cecum abrasion of mice. The mice were imaged at various time points (3, 7, and 14 days) after surgery to test the retention of the HPE-PVA hydrogels in their bodies. Isoflurane (2.5%) was used as the anesthetic agent during imaging. The excitation filter was set at 788 nm, and emission filter was set at 808 nm. The region of interest (ROI) analysis was selected, normalized to the initial value, and plotted over time to evaluate the retention of the HPE-PVA hydrogels. The fluorescent intensity of HPE-PVA hydrogels was visible even 14 days after the injections were administered. These results indicated that the retention time of the HₕPE-PVA hydrogels was even longer (over 14 days).

### In Vivo Biocompatibility of HPE-PVA Hydrogels

The HPE-PVA hydrogels were further evaluated for their in vivo biocompatibility in mice. Tissue sections of major organs, including the heart, liver, spleen, lungs, and kidneys, were collected and stained by H&E. The harvested specimens were fixed with 4% paraformaldehyde (PFA) solution, dehydrated using ethanol, and embedded into paraffin. Then the paraffin-embedded specimens were sectioned into 5 μm thick sections. Subsequently, the sections were stained with H&E trichrome staining. The images were examined blinded. No obvious degeneration, necrosis, lesion, or ischemia of tissues and organs was observed, indicating no inflammatory reaction products after 14 days of treatment.

### Proteomics Analysis

For quantitative proteomics, 100 micrograms of protein from four biological replicates per group (Sham, Injury only, HₕPE-PVA, and Seprafilm®) were taken. Samples were reduced and alkylated, followed by detergent removal using chloroform/methanol extraction. The precipitated pellet was resuspended in 50 mM ammonium bicarbonate and digested with MS-grade trypsin (Pierce). An overnight trypsin digestion at 37° C. was performed in a 1:20 ratio. Digested peptides were cleaned with PepClean™ C18 spin columns (Thermo Scientific)) and re-suspended in 2% acetonitrile (ACN) and 0.1% formic acid (FA) for injection. 1 μg of each sample was loaded onto Acclaim™ PepMap™ 100 75 μm×2 cm C18 LC trap columns (Thermo Scientific) at a flow rate of 4 μl min⁻¹. Peptides were separated on an Easy-Spray PepMap™ RSLC C18 75 μm×50 cm C-18 2 μm column (Thermo Scientific) on a RSLC UltiMate™ 3000 (Thermo Scientific). A step gradient of 4-25% solvent B (0.1% FA in 80% ACN) from 10-100 minutes and 25-45% solvent B for 100-130 minutes at 300 nl min⁻¹ and 50° C. with a 155 minute total run time was applied. Eluted peptides were analysed by an Orbitrap Exploris™ 480 (Thermo Scientific) mass spectrometer in a DDA mode. A survey full scan MS (from m/z 375-1200) was acquired from the orbitrap, with a resolution of 60,000. The automatic gain control (AGC) target for precursor ion scan (MS1) was set at 300%, and the ion filling time was set at 25 ms. The most intense ions, with charge state of 2-6, were isolated in 3 second cycles and fragmented using higher-energy collisional dissociation fragmentation with 30% normalized collision energy and were detected at a mass resolution of 15,000 at 200 m/z. A custom 50% AGC target was set for MS/MS, and the ion filling time was set on auto. The dynamic exclusion was set for 20 seconds with a 10 ppm mass window. Protein identification was performed on PEAKS® studio (Bioinformatics Solutions Inc.) against the swiss-prot Mus musculus protein database downloaded on Febuaray April 2022 using the built-in search engine with default search parameters. The search was set up for full tryptic peptides with a maximum of two missed cleavage sites. Acetylation of protein N-terminus, deamidation (NQ); pyro-glu from Q, pyro-glu from E, and oxidized methionine were included as variable modifications, and carbamidomethylation of cysteine was set as a fixed modification. The precursor mass tolerance threshold was set at 10 ppm, and the maximum fragment mass error was 0.02 Da. The result was filtered and validated using a false discovery rate of 1%. Quantitative data analysis was performed using progenesis QI proteomics 4.2 (Nonlinear Dynamics). Statistical analysis was performed using ANOVA and The Benjamini-Hochberg (BH) method was used to adjust p values for multiple-testing caused false discovery rate. The adjusted p≤0.05 was considered as significant. Various plots such as Heatmap, volcano plot, Venn diagram and PCA was generated using Partek Genomics Suite 7.0.

### Results

### Synthesis and Characterization of HPE Microgels

HPE microgels were prepared as described herein. Here, a 12.5% molar percentage of EGCG to PBA was selected to form a PBA-EGCG complex by reacting in DMSO (Table 1). The formed EGCG-PBA complex served as a dynamic cross-linker for reacting with HA in a 1:1 ratio (PBA:COOH group in HA) in the presence of 4-(4,6-dimetnoxy-1,3,5-triain-2-yl)-4-methylmorpholinium chloride (DMTMM), which is a coupling agent, to synthesize the HPE microgels (HA-PBA-EGCG). In the ¹HNMR spectra, the signal peaks of 3.68-3.08 and 1.83 ppm are assigned to CH₃ groups of HA residues. The signal peaks at 7.6 and 7.31 ppm belong to CH groups of EGCG, which is consistent with reports (Liu, et al. (2017) Biomacromolecules 18 (10): 3143-3155). The signal peaks at 7.78-7.71 and 7.49-7.39 ppm belong to CH groups of PBA residues. The results demonstrate the successful synthesis of HPE with a low molecular weight HA (HₗPE) and a high molecular weight HA (HₕPE). The characteristic peaks for EGCG, PBA, and HA residues were consistent with reports (Lee, et al. (2015) ACS Macro Lett., 4 (9): 957-960; Lee, et al. (2015) Polym. Chem, 6:4462). Based on the special signal peaks of EGCG, PBA, and HA, the actual molar percentages of EGCG and PBA related to HA were calculated (Table 1). For HₗPE, the percentages of EGCG and PBA were 22.8% and 23.8%, respectively. For HₕPE, the percentages of EGCG and PBA were 26.9% and 34.5%, respectively. Fourier transform infrared spectroscopy (FTIR) spectra also confirmed the successful synthesis of HPE. The characteristic peaks of HₕPE were ascribed to 3000 and 3600 cm⁻¹ for O—H stretching vibrations, 2895 cm⁻¹ for C—H stretching vibrations, 1035 cm⁻¹ for C—O stretching vibrations of HA, and 1360 cm⁻¹ for C—H deformation vibration. There were obvious characteristic peaks of PBA at 818 and 715 cm⁻¹ (Ar—H deformation vibration of the aromatic ring). The peaks at 1521˜1429 cm⁻¹ were attributed to aromatic ring deformation (Kim, et al. (2020) Biomaterials 242:119905). The peak of 1560 cm⁻¹ was assigned to C—O stretching vibrations of HA residues. HₕPE exhibited new peaks at 1713 cm⁻¹ and 1206 cm⁻¹, which were assigned to ester C—O stretching vibrations and aromatic C—O stretch of EGCG residues (Cano, et al. (2019) J. Controlled Rel. Soc., 301:62-75; Zhang, et al. (2020) Food Bioprocess Tech., 13 (5): 807-817). These results demonstrated that HₕPE was successfully synthesized.

TABLE 1
Compositions of the Synthesized HₗPE and HₕPE.

| | HA | | | | |
|---|---|---|---|---|---|
| | molecular | Feeding ratio relative | | Actual ratio relative | |
| | weight | COOH (% HA) | | COOH (% HA) | |
| Sample | (kDa) | EGCG | PBA | EGCG | PBA |
| HₗPE | 40 | 12.5 | 100 | 22.8% | 23.8% |
| HₕPE | 900 | 12.5 | 100 | 26.9% | 34.5% |

The morphology and size distribution of the microgels was then evaluated. The representative scanning electron microscopy (SEM) images of the HₗPE and HₕPE microgels showed that the particle size of HₗPE microgels in the dry state varied between 200 and 1000 nm, and the mean size was 562 nm (FIG. 1A). The mean size for the HₕPE microgels was 594 nm, with the size distribution ranging from 200 to 900 nm (FIG. 1B). Both microgels had spherical-like morphologies. FIG. 1C shows that the zeta potentials of HₗPE and HₕPE microgels were −30 and −41 mV, respectively. The cytocompatibility of the HPE microgels was assessed via a CCK-8 assay and Live/Dead™ imaging after culturing L929 fibroblasts for 3 days. After 1 and 3 day incubations, there was no obvious effect on the proliferation of L929 cells in the presence of multiple concentrations of either microgel (FIG. 1D). Live/Dead™ staining also confirmed that most cultured cells were viable after the treatment with the microgels (FIG. 1E). Furthermore, the blood compatibility of HPE microgels was evaluated by measuring the human red blood cell (RBC) lysis during their contact with the microgels. The HPE microgels exhibited less than 1% hemolysis ratios (FIG. 1F), indicating excellent hemocompatibility. All the results above demonstrated that the HPE microgels, with microsized structures and good cyto- and hemocompatibility, are safe for further in vivo application.

### Synthesis and Characterization of HPE-PVA Hydrogels

The injectable catechol-containing cream hydrogels were fabricated on the basis of the boronic ester dynamic covalent bond between HPE microgels and PVA. As shown in FIG. 2B, the HₗPE and HₕPE could be resuspended in water, where they formed milky white emulsions. After complete equilibration in water, the HPE microgels were mixed with PVA by using a 200 μL pipet tip. The cream hydrogels formed after about 10 seconds at room temperature (FIG. 2B). Subsequently, the lyophilized hydrogel samples were also subjected to SEM to obtain the morphological characteristics, and highly porous and interconnected structures with microparticles were observed (FIG. 2C). The multiporous structure with microparticles might guarantee the attachment to the target tissues and gradual gel degradation. The hydrogels mixed with food color dyes (red and blue) could be easily injected through insulin needles (29 G), and then, the two pieces of hydrogels (red and blue) could self-heal after being in contact with each other for 50 seconds (FIG. 2D). Additionally, the HPE-PVA hydrogel showed that it was able to be sprayed, which is beneficial for the treatment of large wound areas caused by surgery. These results showed the hydrogels had injectability and a self-healing capacity, which are suitable for practical application in abdominal antiadhesion in vivo.

The degradation profiles of the HPE-PVA hydrogels in the phosphate buffer solution (PBS) at a pH of 7.4 were evaluated in vitro. The mass of the hydrogels gradually decreased with increasing incubation time (FIG. 2E) due to the reversible boronic ester bond (Zhao, et al. (2021) Adv. Funct. Mater., 31:2009442; Ding, et al. (2021) Adv. Funct. Mater., 31:2011230). The degradation rate of the HₕPEPVA hydrogels (with more than 80% remaining on day 7) was slower than that of the HₗPE-PVA hydrogels (with less than 30% remaining on day 7). The slow degradation rate for HₕPEPVA may be attributed to the enhanced cross-linking density of the hydrogel networks with high molecular weight HA. These results indicated that the HₕPE-PVA hydrogel has a longer retention time and may be more efficient as an antiadhesion material.

The viscoelastic properties of the cream hydrogels were further characterized by rheological tests to evaluate the injectability and self-healing behavior of HPE-PVA hydrogels. As shown in FIG. 2F, time-sweep curves of HPE-PVA hydrogels showed that the G′ (the elastic storage modulus) values were dominant over the corresponding G″ (the viscous loss modulus), indicating the formation of hydrogels with a stable, solid-like state. Moreover, both G′ and G″ values for each hydrogel were kept constant over the whole testing time, indicating good mechanical stability of the formed HPE-PVA hydrogels. The mechanical stability of the hydrogels was reflected by the G′ value. Angular frequency-dependent oscillatory sweeps in the linear viscoelastic regime showed that G′ was dominant over G″ over the full angular frequency range (FIG. 2G). This also demonstrated that the cream hydrogels maintained a solid-like behavior over the entire frequency range, due to the formation of the boronic ester dynamic covalent bond inside the networks of the hydrogels (Tong, et al. (2021) Acta Biomater., 122:111-132). In the strain-dependent oscillatory rheological measurement, the cream hydrogels maintained a solid-like behavior in the low strain (10%) range, with G′ higher than G″, but they adopted a more viscous behavior in the high strain region (600%), where G′ became lower than G″ (FIG. 2H). Both cream hydrogels could restore their original G′ levels immediately after switching from a high strain to a low strain, while both G′ and G″ of the HₗPE-PVA hydrogel were lower than those of the HₕPE-PVA hydrogel. The viscosity of both hydrogels decreased as the shear rate increased, as shown in the shear-rate sweep curves (FIG. 21), confirming their shear thinning behavior. The shearing-thinning behavior of PBA-modified hydrogels was attributed to the disruption and reformation of the dynamic boronic ester bonds between the PBA and the cis-diols groups in the hydrogel networks under shearing (Zeng, et al. (2022) Acta Biomater., 151:210; Kong, et al. (2021) Appl. Mater. Today 24:101090). The hydrogels can be successfully injected through needles several times, and the fragments could self-heal into one intact hydrogel to fully cover and protect the injured tissue surface. Herein, a series of HₕPE-PVA hydrogels consisting of HₕPE microgels and PVA were synthesized by controlling the concentrations of both components. Their viscoelastic properties were also evaluated. Hydrogels with different formulations also showed similar viscoelastic properties, including stable, solid-like states and shear thinning behaviors. However, the higher the concentration ratio of HOPE to PVA, the higher was G′ of the hydrogel. Moreover, the HₕPE-PVA hydrogels with formulations of 2:1 and 3:1 were difficult to extrude through an insulin syringe capped with a 29-gauge needle. Finally, the hydrogel formulation with a 1:1 concentration ratio of HPE:PVA was chosen.

### In Vitro Cytocompatibility and Anti-Cell Adhesion Properties of HPE-PVA Hydrogels

The cytocompatibility of both the HₗPE-PVA and HₕPE-PVA cream hydrogels were also evaluated through in vitro proliferation tests and Live/Dead™ imaging. Both hydrogels exhibited no significant cytotoxicity on L929 cells when the cells were cultured around the hydrogels on 2D plates (FIG. 3A), which was further confirmed through Live/Dead™ staining (FIG. 3B). The cream hydrogels also exhibited no significant hemolysis on human RBCs (FIG. 3C). The adhesion and excessive proliferation of fibroblasts can secrete a great deal of collagen and other extracellular matrices (ECMs), which play important roles in the formation of fibrous adhesion (Zindel, et al. (2021) Science 371 (6533): 993; Tang, et al. (2020) Acta Biomater., 116:84-104). Thus, L929 cells were further seeded on the surface of HPE-PVA hydrogels and cultured them for 18 hours to evaluate the cellular attachment. Confocal images showed that very few cells were observed on the HPE-PVA hydrogel surface, whereas a large number of L929 cells settled and spread on the tissue culture polystyrene (TCPS) surface, showing the low cell adhesion capacity of the hydrogels.

### In Vitro Antioxidative and Anti-Inflammatory Properties of HPE-PVA Hydrogels

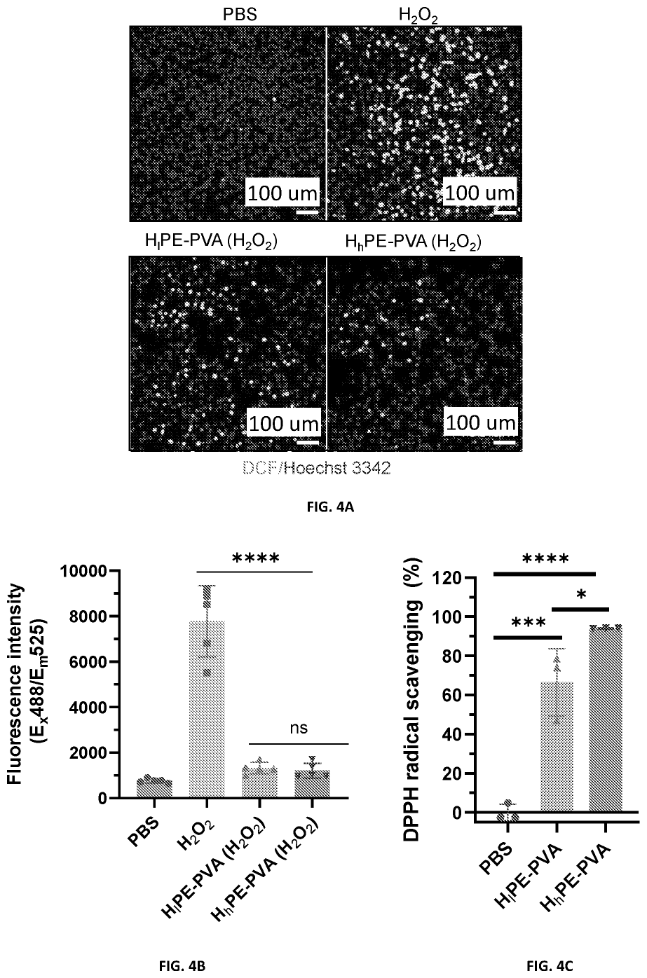

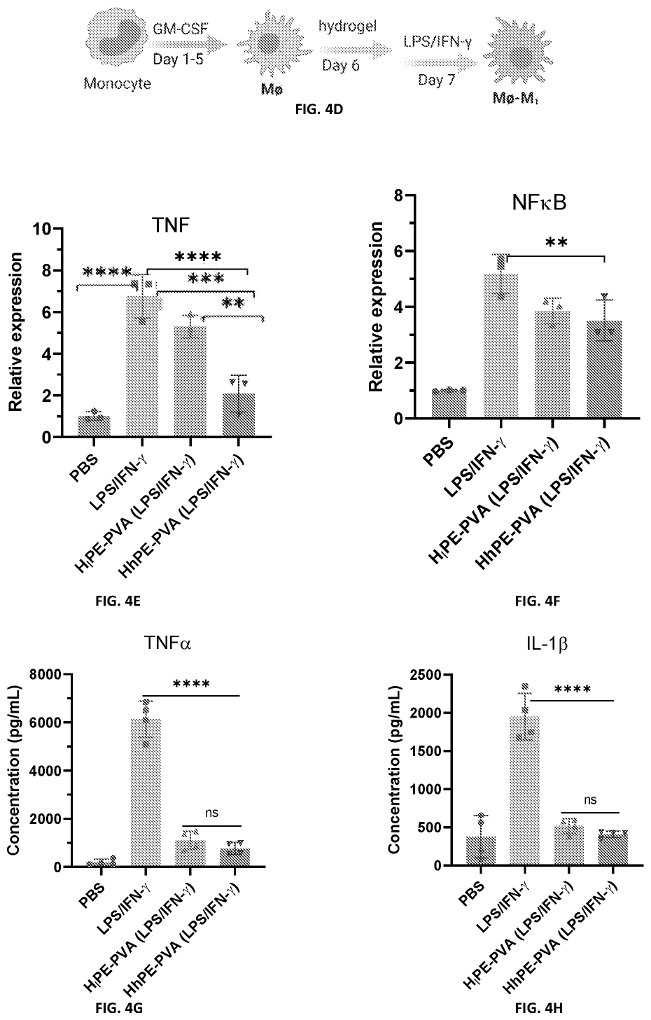

Due to the free phenol groups and the dynamic nature of boronic esters in the HPE-PVA hydrogels, the hydrogels exhibit promising antioxidative properties. The in vitro antioxidative efficiency of the HPE-PVA hydrogels was investigated by using an ROS scavenging test and a 1,1-diphenyl-2-picrylhydrazyl (DPPH) free radical scavenging test. The HPE-PVA hydrogels were incubated with L929 fibroblasts treated with the hydrogen peroxide (H₂O₂), which can stimulate the cells to produce excessive intracellular ROS. Meanwhile, nonfluorescent 2′,7′-dichlorodihydro-fluo-rescein diacetate (H2DCFDA) was used as a probe to detect the intracellular ROS level after oxidation because it converts to highly fluorescent 2′,7′-dichlorofluorescein (DCF) in the presence of ROS. As shown in FIG. 4A, both HₗPE-PVA and HₕPE-PVA hydrogels could quench fluorescence in L929 cells, unlike the positive group (H₂O₂ only). The quantitative analysis of the DCF fluorescence signal intensity showed that HPE-PVA hydrogels could significantly reduce intracellular ROS (FIG. 4B). In addition, to test radical scavenging properties, the HPE-PVA hydrogels were incubated in a DPPH solution. It was found that the HhPE-PVA hydrogels showed a much higher DPPH scavenging efficiency (over 90% radical scavenging) than HlPE-PVA hydrogels (about 70% radical scavenging) (FIG. 4C).

Next, pro-inflammatory M1 macrophages derived from human monocytes were implemented to evaluate the anti-inflammatory effects of the HPE-PVA hydrogels in vitro. As shown in FIG. 4D, monocytes were differentiated into macrophages by following a reported protocol (Liu, et al. (2022) Bioact Mater., 14:61-75). The macrophages were cocultured with HPE-PVA hydrogels and then stimulated to the M1 phenotype using lipopolysaccharide (LPS)/interferon-gamma (IFN-γ). Quantitative real time polymerase chain reaction (qPCR) analysis showed that both tissue necrosis factor (TNF)-α and nuclear factor kappa B (NFκB) expressions were significantly upregulated in M1 macrophages after activation by LPS/IFN-γ and significantly downregulated in the HPE-PVA hydrogel group (FIG. 4E, 4F). The M1 macrophage-secreted inflammatory cytokines, i.e., TNF-α and interleukin (IL)-1beta (IL-1β), were measured by an enzyme-linked immunosorbent assay (ELISA). It was found that TNF-α and IL-1β were significantly increased after stimulation, while the proinflammatory cytokine secretions were significantly decreased after treatment with HPE-PVA hydrogels (FIG. 4G, 4H). These results demonstrated that both HₗPE-PVA and HₕPE-PVA hydrogels had anti-inflammatory effects by downregulating pro-inflammatory gene expressions and cytokine secretions.

### In Vivo Antiadhesion Efficacy of HPE-PVA Hydrogels

A murine cecum-abdominal wall adhesion model was used to evaluate the ability of the HPE-PVA hydrogels to prevent postoperative cecum-abdominal adhesions, with comparisons to commercial Seprafilm® and untreated mice. After injuries to cecum and abdominal wall were induced, 50 μL of the HPE-PVA hydrogels (HₗPE-PVA or HₕPE-PVA) was injected through a syringe and then gently spread around the surfaces of wound areas. The commercial Seprafilm® films were applied directly to the surface of the wound areas. The group with mice treated with only saline (50 μL) was used as the negative control (denoted as Injury group), and the group of mice with an exposure of the cecum and abdominal wall but without injury served as the Sham control.

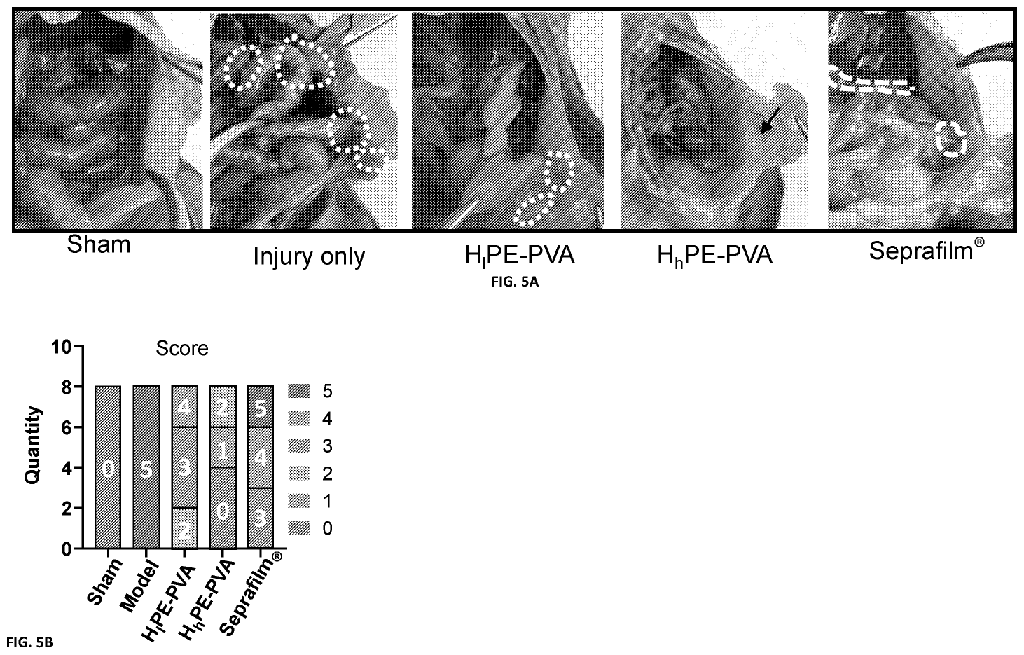

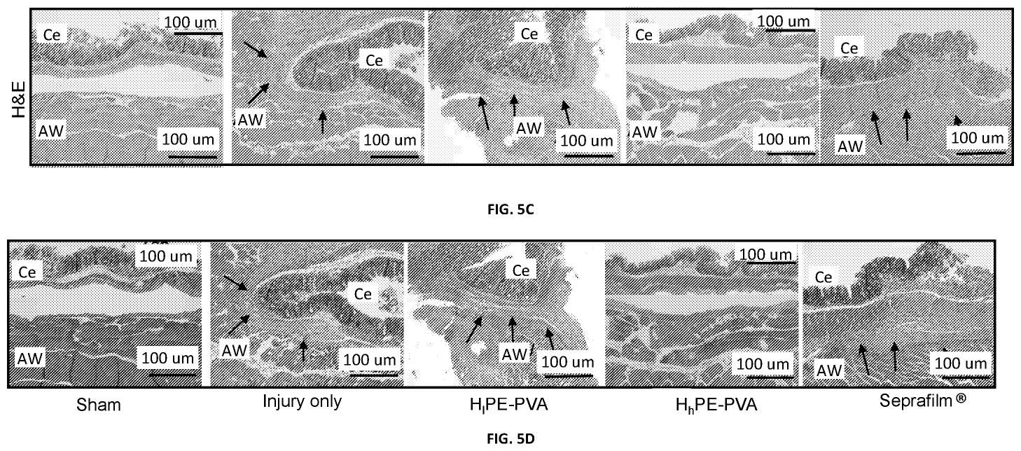

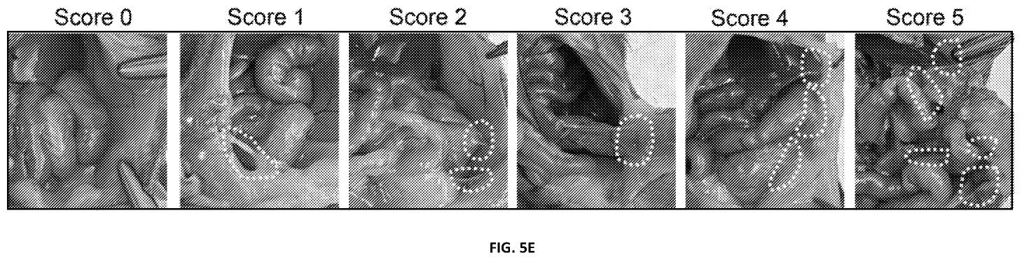

Two weeks after the surgery, a gross assessment of the cecum-abdominal adhesion formation was performed by reopening the abdomen after the mice were euthanized. Before dissecting the adhesions to harvest the cecum and abdominal wall tissues, images were taken and given to blinded graders for examination to determine the level of adhesion scores, which are based on the presence and severity of adhesions according to the standard scoring system from 0 to 5 (FIG. 5E) (De Clercq, et al. (2016) Biomaterials, 96:33-46; Yu, et al. (2021) Adv. Mater., 33 (16): 2008395). The representative images of the observed adhesion formation in five groups are shown in FIG. 5A, and all groups were scored at postoperative day (POD) 14 (FIG. 5B). The Injury-only group showed severe adhesions between the abdominal wall and cecum, with an average score of 5 for all mice (n=8). Such severe adhesions required a sharp dissection to detach the large fibrous tissue surrounding both the cecum and abdominal wall. The adhesive sites in the commercial Seprafilm® membrane group were clearly observed, with an average score of 3.9. This indicated that the Seprafilm® can only reduce the postoperative adhesions to some extent, and the overall efficacy was not satisfactory. The application of HₗPE-PVA hydrogels also alleviated the adhesions, with an average score of 3. The group with HₕPE-PVA hydrogel treatment showed almost no adhesive tissue, with an average score of 0.8. This is comparable to the Sham group, which has an average score of 0, indicating that the adhesions were effectively prevented.

Hemotoxylin and eosin staining (H&E, FIG. 5C) and Masson's trichrome staining (FIG. 5D) were used to histologically visualize the tissue adhesions and healing at the injury sites. As shown in FIG. 5C, severe adhesions occurred between the abdominal muscle layer and the cecum in the Injury-only group (marked by arrows). This group had accumulated connective tissues accompanied by a large number of leukocytes, indicating that the formation of the adhesions was closely related to inflammation. Moderate adhesions with slight bridging between the cecum and the surrounding abdominal wall were also observed in the HₗPE-PVA hydrogels and commercial Seprafilm® groups. In contrast, there was no apparent adhesion between the abdominal wall and the cecum after treatment with HₕPE-PVA hydrogels. Masson's trichrome staining showed that abundant collagen was deposited in the Injury-only group. Despite the protective effects that were provided by the commercial Seprafilm® and the HₗPE-PVA hydrogels, collagen deposition was still obvious, leading to the formation of mild/moderate adhesions. Consistent with the H&E staining, no or very limited collagen deposition was observed in the HₕPE-PVA hydrogel group, demonstrating an excellent antiadhesive effect.

### In Vivo Anti-Inflammatory and Antioxidative Effects of HPE-PVA Hydrogels

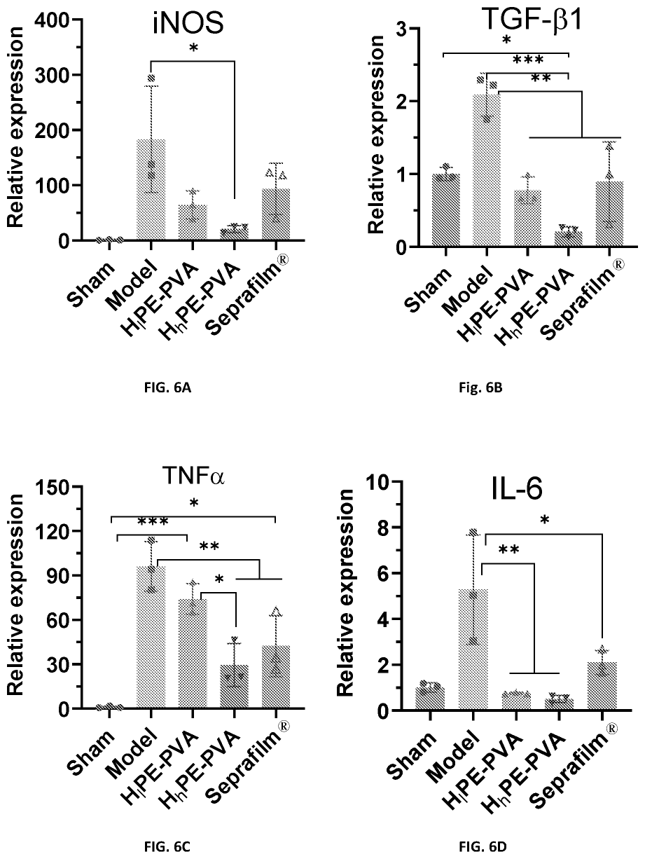

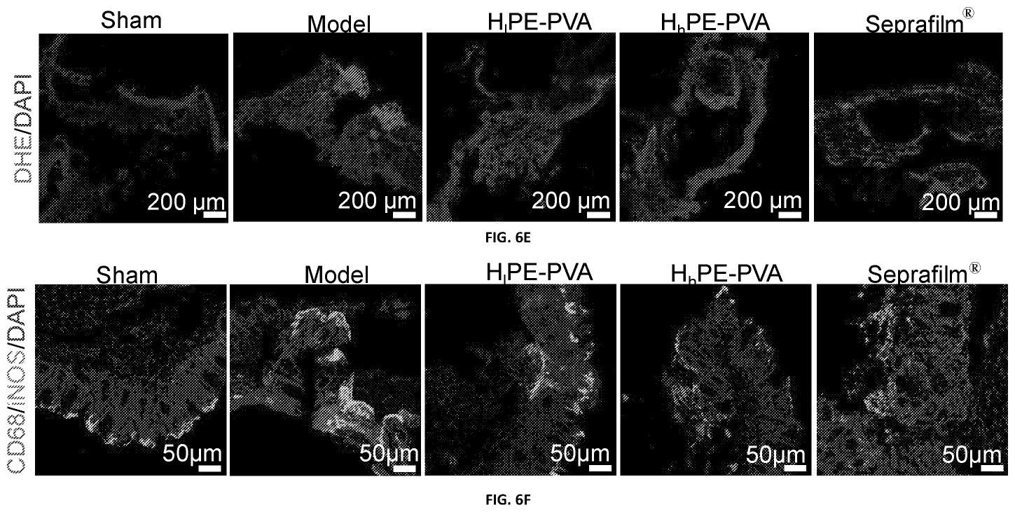

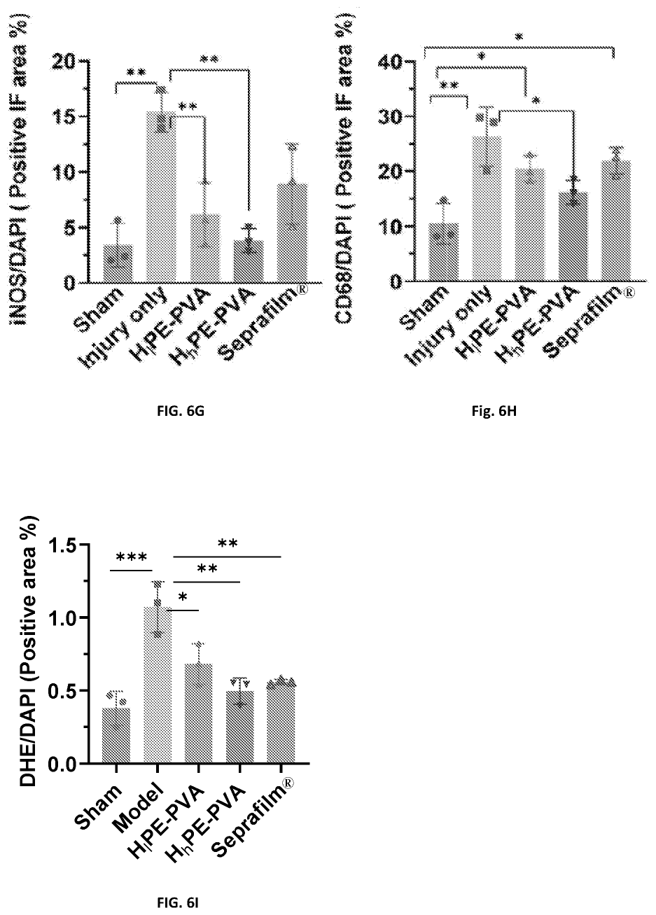

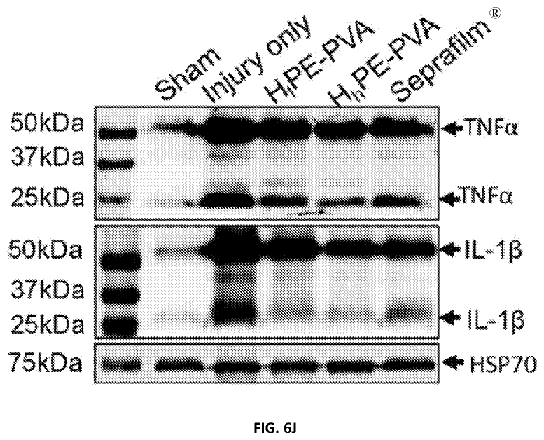

To further investigate the anti-inflammatory and antioxidative effects of the PHE-PVA hydrogels, molecular biology studies were performed by using qPCR, immunofluorescent (IF) staining, and Western blotting methods. Compared to the Sham group, surgical trauma in the Injury-only group significantly increased the pro-inflammatory gene expressions of inducible nitric oxide synthase (iNOS), transforming growth factor (TGF)-β1, TNF-α, and IL-6 (FIG. 6A-6D). After treatment with HₕPE-PVA, the iNOS gene expression was significantly downregulated compared to the Injury-only group, whereas both the HₗPE-PVA hydrogel and the Seprafilm® groups could only slightly reduce the iNOS gene expression, without statistical differences (FIG. 6A). FIG. 6B-6D shows that TGF-β1, TNF-α, and IL-6 genes were significantly downregulated in all the treatment groups and that HₕPE-PVA hydrogels had the best efficacy. IF staining showed that all groups showed positive expressions of iNOS and CD68. INOS has been reported to be expressed in the normal surface of colonic epithelium tissue. The physiological expression of iNOS might act as an oxidative barrier to reduce bacterial translocation and provide defensive functions.42 In the Injury group, increased and unorganized iNOS expression was observed throughout the adhesion regions (FIG. 6E). In contrast, the tissue with the HₕPE-PVA hydrogel treatment maintained a low level of organized iNOS expression on the cecal villi. Semiquantitative ImageJ analysis was conducted to evaluate and compare the fluorescent area ratios of iNOS (pro-inflammatory marker)/DAPI (cell nucleus) in different groups (FIG. 6G). Consistent with the IF staining results, the treatments with the HPE-PVA hydrogels and Seprafilm® reduced the iNOS/DAPI ratios compared to the Injury-only group. The HPE-PVA hydrogels further significantly decreased the ratio, indicating the anti-inflammatory effects. The fluorescent area ratios of CD68/DAPI was also compared in different groups (FIG. 6H). Consistent with the results above, the treatments with HPE-PVA hydrogels could regulate inflammatory reactions. Western blot results showed that the protein expressions of two pro-inflammatory cytokines, TNF-α and IL-1β, were increased in the Injury group, as expected. All three treatments decreased the expressions of these two cytokines, and the HₕPE-PVA hydrogel group showed the highest inhibitory effect on their expressions.

The antioxidative effects of HPE-PVA hydrogels was also investigated in vivo by dihydroethidium (DHE, a ROS prober) staining. As shown in FIGS. 6F and 6H, extensive DHE signals were detected in the Injury-only group, indicating the existence of a large amount of ROS accumulation in the injury and adhesion region. In contrast, the DHE expressions in all the treatment groups were significantly reduced compared to the Injury-only group, indicating that the ROS levels were reduced. FIG. 6H showed that ROS generation exhibited no significant difference among the HₗPE-PVA hydrogel, HₕPE-PVA hydrogel, and Seprafilm® groups.

### In Vivo Fibrinolytic Activity of HPE-PVA Hydrogels

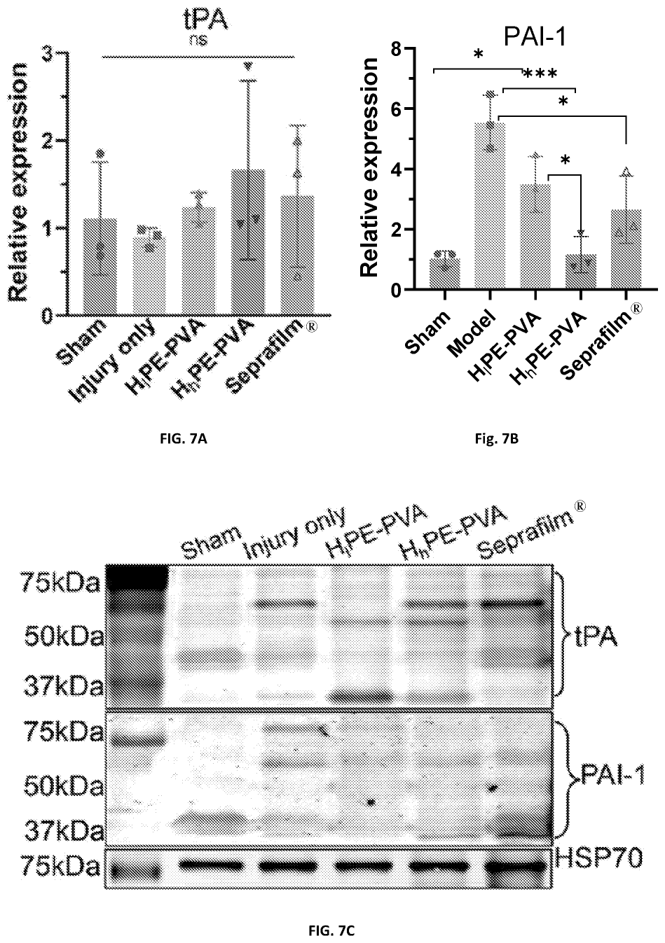

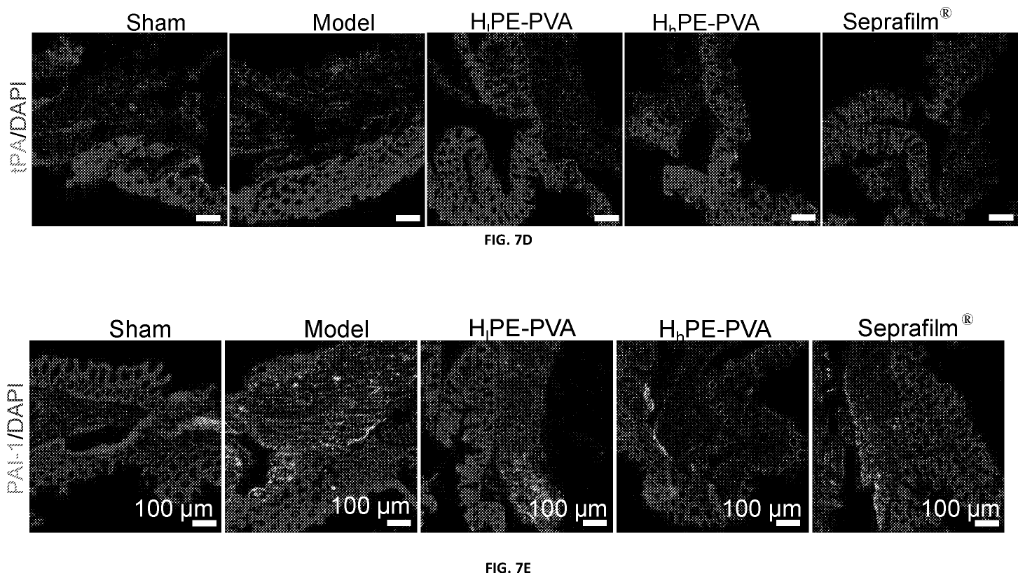

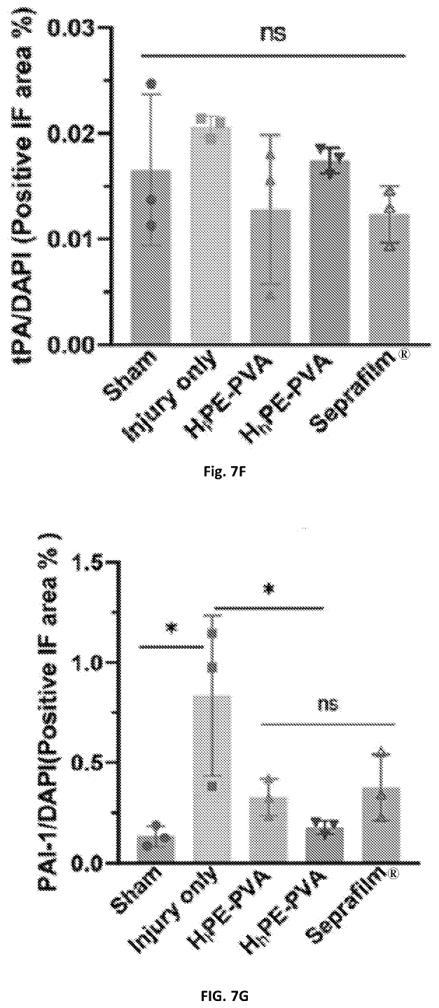

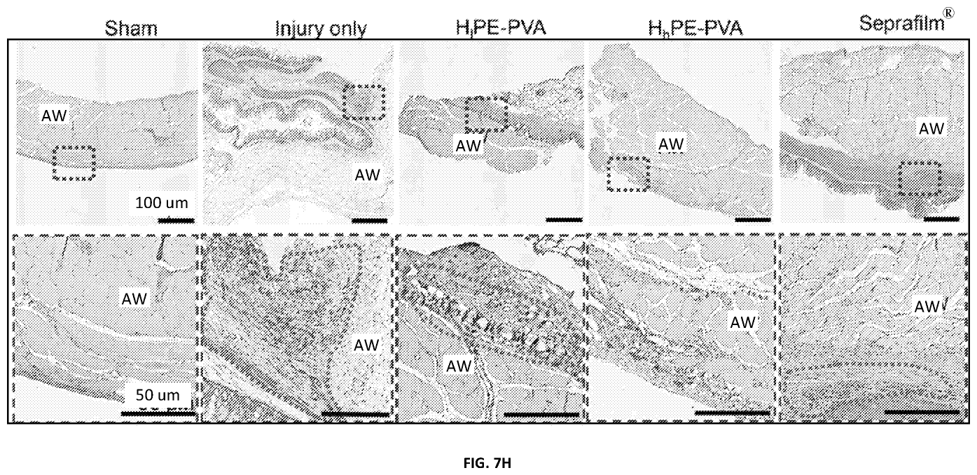

The formation of postoperative adhesions is induced by the imbalance between fiber degradation and deposition (Yu, et al. (2021) Adv. Mater., 33 (16): 2008395; Zhao, et al. (2021) Chem. Eng. J., 404:127096). Tissue plasminogen activator (t-PA) is the key mediator of fibrinolysis, while plasminogen activator inhibitor (PAI)-1 is the major inhibitor of tPA and reduces fibrinolytic activity. Thus, the effects of HPE-PVA hydrogels on the gene and protein expressions of tPA and PAI-1 was studied in the injured tissues at POD 14. The qPCR results showed that the expressions of tPA in different groups were comparable without significant differences (FIG. 7A). However, the level of PAI-1 in the Injury-only group was significantly upregulated compared to Sham group. In all three treatment groups, the expressions of PAI-1 were statistically downregulated compared to that in the Injury-only group, with the lowest expression in the HₕPEPVA hydrogel group (FIG. 7B). Consistent with the gene expression results, the Western blot and IF staining results confirmed that there were no differences in tPA protein expressions among all the five groups (FIGS. 7A, 7D, 7F). Confocal images, shown in FIG. 7E, demonstrated clear PAI-1 expressions, especially in the Injury-only group. FIGS. 7C and 7G confirmed that the three treatment groups had lower PAI-1 expressions compared to the Injury group.

The α-smooth muscle actin (α-SMA) was also an important mesothelial and fibroblast marker in the abdominal adhesions. In the abdominal adhesion sites with fibrotic scars between the abdominal wall and visceral layers, the α-SMA expression appeared to be upregulated (Zindel, et al. (2021) Nat. Commun., 12 (1): 7316). Immunohistochemistry (IHC) staining of α-SMA was performed and is shown in FIG. 7H. In the Injury-only group, the α-SMA expression was obvious in the adhesive site, whereas the expressions of α-SMA were reduced after the treatment with the HPE-PVA hydrogel and Seprafilm®, especially in the HₕPE-PVA hydrogel group.

### In Vivo Retention and Biocompatibility of the HPEPVA Hydrogel

To confirm the retention time of hydrogels on the wound areas of the cecum and abdominal wall, fluorescent dye Cy7.5-labeled HPE-PVA hydrogels were applied in mice. The fluorescent imaging permits monitoring of the retention time by an IVIS system (PerkinElmer) in vivo. On day 3 and 7, the fluorescent signals were clearly detected for both HPE-PVA hydrogel groups. Although the fluorescent signals were still visible on day 14, the fluorescent intensity was significantly decreased, indicating the degradation/dissociation of the hydrogels. The fluorescent intensity for HₕPE-PVA was stronger, indicating that the retention time of the HₕPE-PVA hydrogels was longer (over 14 days). The in vivo retention time for the HₕPE-PVA hydrogels is sufficient to prevent the adhesions, while the hydrogels gradually degrade to guarantee that the wound healing is not interrupted. The HPE-PVA hydrogels were further evaluated for their in vivo biocompatibility in mice. Tissue sections of major organs, including the heart, liver, spleen, lungs, and kidneys, were collected and stained by H&E. There was no obvious degeneration, necrosis, lesions, or ischemia of tissues or organs, indicating no inflammatory reaction products after 14 days of treatment. These results were consistent with those obtained in vitro and indicated that the hydrogels had good in vitro and in vivo retention times and biocompatibility.

### Proteomics Analysis

Next, proteomics was used to examine and compare differential protein expression profiles. Principal component analysis (PCA) showed that the Sham control group could be clearly differentiated from the Injury-only and Seprafilm® groups. Some overlap between the Sham and HₕPE-PVA hydrogel groups was also observed. Proteomics analysis allowed an overall identification of 6603 proteins. 2882 of these proteins were expressed in all four groups, while 109 and 316 proteins were uniquely expressed in the Sham and Injury-only groups, respectively. Top differentially expressed proteins were selected based on fold change (FC≥2) and a p-value <0.05. Among these, many proteins are reported to be related to inflammation, immune cell recruitment and infiltration, fibrosis, and ECM remodeling. For example, lymphocyte cytosolic protein (LCP) 1 (known as plastin-2) is exclusively expressed in leukocytes, such as macrophages and granulocytes, and is related to macrophage and neutrophil activation (Shinomiya, H. (2012) Int. J. Cell Biol. 2012:213492). Serine protease inhibitor (serpin) a3n, a member of the serpin superfamily, is reported to play an important role in wound healing and the fibrotic process (Hsu, et al. (2014) Cell Death Dis., 5: e1458; Gong, et al. (2020) Biochem. Biophys. Res. Commun., 532 (4): 598-604). Collagen beta (1-O)-galactosyltransferase (Colgalt) 1 is responsible for proper galactosylation of hydroxylysine residues in many types of collagens (Col). It has been reported to regulate the remodeling of Col IV (Geister, et al. (2019) Dis. Model Mech., 12 (6): dmm037176; Liu, et al. (2022) Eur. J. Med. Res., 27 (1): 122), which is one of the major ECM components in the mesothelium. Consistent with several studies (Sandoval, et al. (2016) J. Pathol., 239 (1): 48-59; Foster, et al. (2020) Nat. Commun., 11 (1): 4061), many ECM (like Col I, III, and IV and fibrin) and epithelial/mesothelial to mesenchymal transition (EMT/MMT) (like vimentin, αSMA, and tenascin) related proteins were significantly upregulated in the Injury-only group.

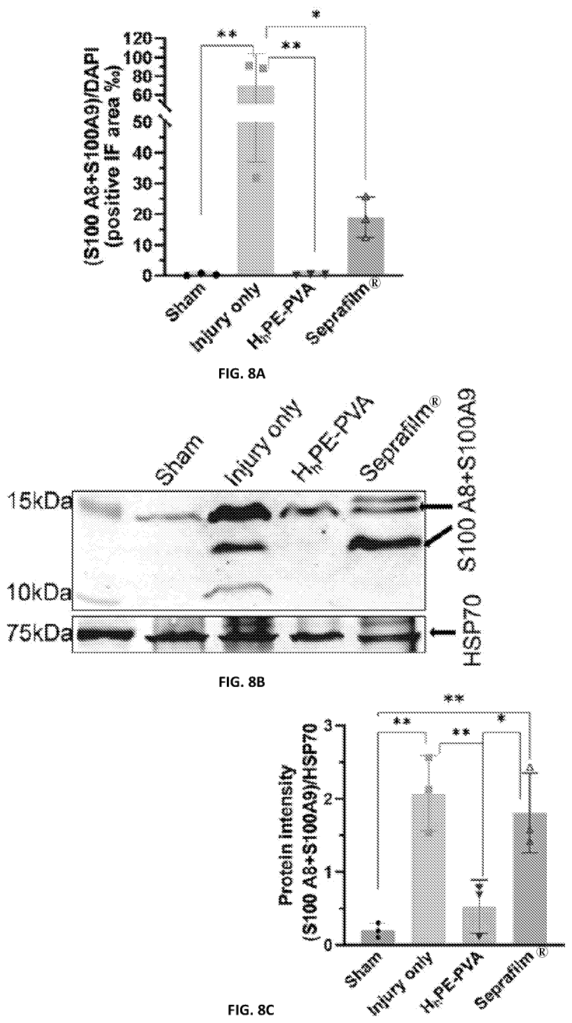

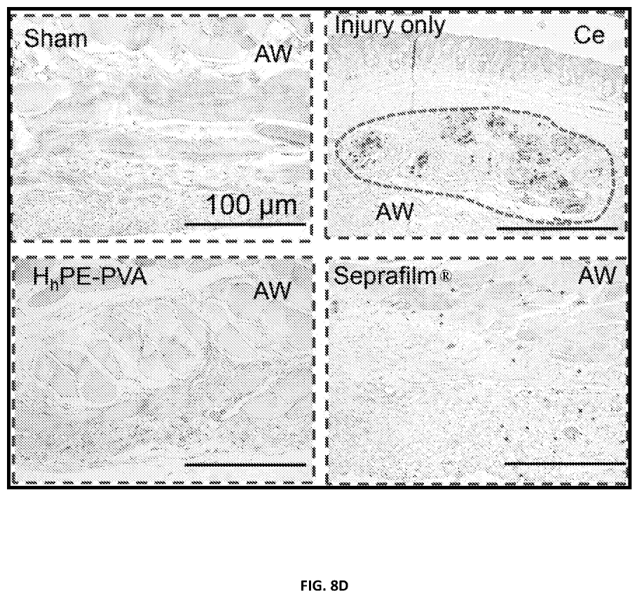

The volcano plot of the Injury-only and HhPE-PVA hydrogel groups revealed dramatic protein changes with 486 proteins upregulated and 48 proteins downregulated in the Injury-only group compared to the HhPE-PVA hydrogel group. The differentially expressed proteins from the proteomics analysis were further analyzed using Ingenuity Pathway Analysis (IPA). The graphical summary in the IPA demonstrated/predicted the key entities and their connections. Among the upregulated proteins, S100A8 and S100A9 (also known as calgranulin A and B or myeloid-related protein [MRP]8 and -14, respectively), which are Ca2+ binding proteins and are inflammation-associated proteins expressed in many inflammation-related cells, including neutrophils, macrophages, epithelial cells, and fibroblasts, were of note (Wang, et al. (2018) Front. Immunol., 9:1298; Zhong, et al. (2016) Am. J. Pathol., 186 (1): 109-122). As shown in FIG. 8A, IF staining showed that the expression of S100A8+S100A9 increased significantly in the Injury group, while the expression of S100A8+S100A9 was significantly reduced after treatment with HₕPE-PVA hydrogel. Western blot results further confirmed that the S100A8+S100A9 levels in traumatic sites were significantly lower in the HₕPE-PVA and Sham groups than those in the Injury-only group (FIGS. 8B and 8C). IHC staining also showed that the expression of S100A8+S100A9 was significantly reduced after treatment with the HₕPE-PVA hydrogel, compared with the Injury-only group (FIG. 8D). IPA network analysis also predicted S100A8 and S100A9 as one of the key central hubs. The top 5 canonical pathways identified by IPA analysis are: eukaryotic initiation factor 2 (eIF2) signaling (p-value: 7.41E-15; overlap: 13.2% (30/227)); protein ubiquitination pathway (p-value: 1.73E-10; overlap: 9.9% (27/274)); clathrin-mediated endocytosis signaling (p-value: 5.51E-10; overlap: 11.4% (22/193)); mTOR signaling (p-value: 2.01E-8; overlap: 9.8% (21/214)); and phagosome maturation (p-value: 2.07E-8; overlap: 11.4% (18/158)).

Herein, multifunctional HPE-PVA cream hydrogels were synthesized and characterized for intra-abdominal antiadhesive therapy. The cream hydrogel system was synthesized based on the boronic ester dynamic covalent bond between HPE microgels and PVA. The incorporation of HPE microgels has several advantages. First, both EGCG and PBA provide multifunctionality, like antioxidation and anti-inflammation (Shin, et al. (2019) Adv. Funct. Mater., 29 (43): 1903022; Zhao, et al. (2021) Adv. Funct. Mater., 31:2009442; Kim, et al. (2020) Biomaterials 242:119905; Chen, et al. (2022) ACS Nano 16 (2): 2429-2441). Second, the catechol groups in EGCG enable the formation of two reversible boronic ester bonds with PBA, which may act as a dynamic cross-linker for further hydrogel cross-linking. Third, the microgels can act as fillers in polymer hydrogels to provide a more dynamic network structure associated with enhanced self-healing (Bertsch, et al. (2023) Chem. Rev., 123:834). Furthermore, the microgels can serve as a drug delivery system to effectively and sustainedly release encapsulated payloads. As expected, the HPE microgels were cytocompatible and hemocompatible without impairing fibroblast cell viability and proliferation and without causing any hemolysis when directly contacting cells and blood.

The HPE-PVA cream hydrogels were facilely synthesized by simply mixing HPE microgels and a PVA solution at room temperature. The overall preparation process of the HPE-PVA hydrogels is straightforward and without any toxic reagents or any complicated chemical reactions, which enables practical use in clinical applications. Ideal hydrogel antiadhesion barrier materials should be capable of viscous flow when shear or extrusion is applied, enabling them to completely cover the target tissues (Yin, et al. (2021) Adv. Funct. Mater., 31 (41): 2105614). Thus, the physical parameters should be modulated first by the alternation of the HPE-PVA hydrogel formulation to obtain an ideal adhesion barrier. FIG. 2F shows that the HPE-PVA hydrogels based on this formulation demonstrated shear-thinning fluid behaviors. An appropriate retention time is another important requirement for an effective antiadhesion material. Abdominal adhesion formation generally started at days 3-7 after surgery, and then, a dense adhesion was formed at days 7-14 (Cheong, et al. (2001) Human Reproduction Update 7:556-566). Thus, the retention time of antiadhesion materials should cover these time frames. The current results showed that the retention time of the HₕPE-PVA hydrogel is over 14 days in vitro and in vivo, indicating that the HₕPE-PVA hydrogel would be beneficial for effective adhesion prevention. Similar to HPE microgels, the HPE-PVA cream hydrogels exhibited a good cytocompatibility with L929 fibroblasts and hemocompatibility with human RBCs in vitro. Another important feature of biomaterial-based abdominal antiadhesive treatments is antioxidative and anti-inflammatory properties, which have not been achieved by commercial products or most preclinically tested materials (Chandel, et al. (2021) Macromol. Biosci., 21 (3): 2000395). Although anti-inflammatory drugs could be loaded within the antiadhesion barrier materials (Guo, et al. (2020) Chem. Mater., 32 (15): 6347-6357; Zhang, et al. (2021) J. Controlled Release 335:359-368), the drug dose and release profile have to be well designed, which increases the overall complexity. The results showed that the antioxidant efficiency of L929 cells was significantly enhanced after their treatment with HPE-PVA hydrogels and that the radical clearance rate was higher than 90%. Such antioxidative properties were attributed to the incorporation of EGCG and PBA components. The HPE-PVA hydrogels also had excellent anti-inflammatory activities that are able to downregulate the proinflammatory related gene expressions and cytokine secretions of M1 macrophages. Without being bound by theory, the molecular mechanisms of such anti-inflammatory effects may be due to the inhibitory action of EGCG on several pro-inflammation related signaling pathways, like NFκB and phosphoinositide 3-kinase (PI3K)/Akt/mammalian target of rapamycin (mTOR) pathways (Lakshmi, et al. (2020) Arch. Biochem. Biophys., 695:108620; Peairs, et al. (2010) Cell Mol. Immunol., 7 (2): 123-132).

The formation of abdominal adhesions involves a series of coordinated events and mechanisms, like injury, wound healing, fibrinolysis, and the fibrotic process, and oxidation and inflammation are the key factors (Tang, et al. (2020) Acta Biomater., 116:84-104; Cheong, et al. (2001) Human Reproduction Update 7:556-566). How the antioxidative and anti-inflammatory HPE-PVA hydrogels affected all these processes was evaluated in comparison with a commercial product (i.e., Seprafilm®). The in vivo cecum-abdominal adhesions were first successfully induced using a sidewall defect and cecum abrasion model in mice. Moderate adhesions in the commercial Seprafilm® group were formed, which is consistent with reports that show a substantial amount of tissue remaining on the wound site surface as a result of tight adhesions (Stapleton, et al. (2019) Nat. Biom. Engr., 3 (8): 611-620; Zhou, et al. (2022) ACS Nano 16:7636). This might be because the membranes from Seprafilm® were applied as a solid sheet and could not completely cover the traumatized surfaces. Additionally, the Seprafilm® membrane might be randomly attached to the surface of injured tissues (Gao, et al. (2022) Adv. Mater. Interfaces 9:2200063). The mice treated with HₕPE-PVA did not suffer from adhesions, and the defects were almost repaired within 2 weeks. Interestingly, although both HPE-PVA hydrogels were easily applied to fit the irregular surfaces of the wounds, the HₗPE-PVA hydrogel was not as effective as its high molecular weight counterpart (i.e., HhPEPVA) (FIG. 5B), showing mild to moderate adhesions. The differences between HPE-PVA hydrogels with low (40 kDa) and high (600-1200 kDa) molecular weight HA were evaluated in vitro and in vivo. The HₕPE-PVA hydrogel had slightly better antioxidative (FIG. 4C) and anti-inflammatory (FIGS. 4E and 6C, 6H) effects. HₕPE-PVA hydrogels were also more stable in vitro and in vivo. In addition, high molecular weight HA (>500 kDa) has anti-inflammatory effects, whereas low molecular weight HA (10-500 kDa), in contrast, promoted the production of inflammatory mediators (Rayahin, et al. (2015) ACS Biomater. Sci. Eng., 1 (7): 481-493). Regardless of the reason, HₕPE-PVA hydrogels had better antiadhesion efficacy.

After injury, the balance between fibrin formation and fibrinolysis is disrupted by excessive coagulation and inflammatory responses (Kosaka, et al. (2008) Nat. Med., 14 (4): 437-441). TPA and its inhibitors (like PAI-1) are important parts of the fibrinolytic system. In response to pro-inflammatory cytokine release, more PAI-1 was secreted by mesothelial and other cells to inhibit the activation of plasminogen, which in turn stimulated macrophages to enhance inflammation and adhesion (Cheong, et al. (2001) Human Reprod. Update 7:556-566; Arjmand, M. H. (2022) Arch. Physiol. Biochem., 128 (4): 869-874). The results showed that all the antiadhesion treatments could significantly downregulate the expressions of PAI-1 at both the gene and protein levels, while the HhPE-PVA hydrogel group had the lowest expression of PAI-1. However, no significant differences in expression levels of tPA were observed between all groups. This was inconsistent with some studies, which reported increased tPA expression for the antiadhesion treatments (Yu, et al. (2021) Adv. Mater., 33 (16): 2008395; Gao, et al. (2022) Adv. Mater. Interfaces 9:2200063). The difference may be because pro-inflammatory processes also stimulate tPA expression (Lin, et al. (2014) Am. J. Clin. Exp. Immunol., 3 (1): 30-36). The anti-inflammatory effects of HPE-PVA hydrogels might counteract the protective effect induced by an increased tPA expression, resulting in comparable tPA levels.

The proteomics analysis identified the alterations in protein profiles after the abdominal adhesions and demonstrated the similarity between the HₕPE-PVA hydrogel treatment group and the Sham group. Among various upregulated inflammation-associated proteins, attention has been drawn to S100A8 and S100A9, which are Ca2+ binding proteins that play critical roles in rearranging the cytoskeleton, stimulating leukocyte recruitment, and inducing cytokine secretions. S100A8 and S100A9 are key factors in inducing tissue fibrosis (Araki, et al. (2021) J. Mol. Med. (Berl) 99 (1): 131-145). Ca2+ signaling is a potential molecular trigger for initiating abdominal adhesions and that Ca2+ signaling inhibitors can block early adhesion (Fischer, et al. (2020) Nat. Commun., 11 (1): 3068). S100A8+S100A9 may be involved in these processes. In addition, S100A8+S100A9 may indirectly activate JUN and mTOR pathways (Yi, et al. (2022) Cells 11 (12): 1911; Foster, et al. (2020) Nat. Commun., 11 (1): 4061).

In summary, injectable cream-like hydrogels were designed and synthesized by the conjugation reaction of HPE microgels and PVA based on the dynamic boronic ester bond. The HPE-PVA hydrogels had multiple functionalities, including rapid gelation, self-healing, antioxidation, anti-inflammation, and anti-cellular adhesion. The HₕPE-PVA hydrogels (with high molecular weight HA) showed superior antioxidative and anti-inflammatory properties in vitro and in vivo compared to their counterparts with low molecular weight HA. The HₕPE-PVA hydrogels also had longer in vivo retention time and significantly reduced the abdominal adhesion formation in a mouse model with cecum-abdominal wall adhesion injury, compared to the commercial Seprafilm® group or Injury-only group. Label-free quantitative proteomics analysis demonstrated that S100A8+S100A9 expressions were associated with adhesion formation, and the HₕPE-PVA hydrogels significantly downregulated the S100A8+S100A9 expressions. The microgel-containing hydrogels with multifunctionality demonstrate their clinical application in preventing postoperative adhesions.

### Example 2

Zwitterionic polymers are polymers which contain both positively and negatively charged groups in their repeating units and exhibit overall neutral charge. Zwitterionic materials hold water more strongly through ionic solvation. This strong hydration effect constitutes the foundation of a series of exceptional properties of zwitterionic materials, such as resistance to protein adsorption and lubrication at interfaces. A degradable multifunctional cream zwitterionic hydrogel was synthesized based on hyaluronic acid, 3-[[2-(acryloyloxy)ethyl](dimethyl) ammonio]-1-propanesulfonate (SPDA), PBA, EGCG, and PVA. By using similar approach, other zwitterionic monomers could be conjugated to HA.

Generally, the zwitterionic cream HSPE-PVA hydrogels with HA-SPDA-PBA-EGCG (HSPE) microgels and PVA were synthesized as described above. Briefly, the PBA-EGCG complex was synthesized by mixing PBA and EGCG in DMSO at room temperature. The formed PBA-EGCG complex was directly processed for the conjugation with HA-SPDA in the presence of DMTMM to generate the HSPE microgel at RT for 3 days. When the prepared HSPE microgels were mixed with an aqueous PVA solution, the zwitterionic HSPE-PVA hydrogels were immediately fabricated within about 10 seconds.

To make HA-SPDA, thiolated HA (HA-SH) was first synthesized. Briefly, HA (600-1200 kDa) was reacted with 3,3′-dithiobis(propanoic dihydrazide) (DTP) in the presence of 1-ethyl-3-(3-dimethylaminopropyl) carbodiimide (EDC) and hydroxybenzotriazole (HOBt) at pH 4.7 and then dithiothreitol (DTT), pH 8.5. ¹H NMR spectra of HA and HA-SH confirmed the presence of HA-SH with the signal peak at δ=3.0˜2.5 ppm attributed to the CH₂ groups of DTP residues. A schematic of the synthesis is provided.

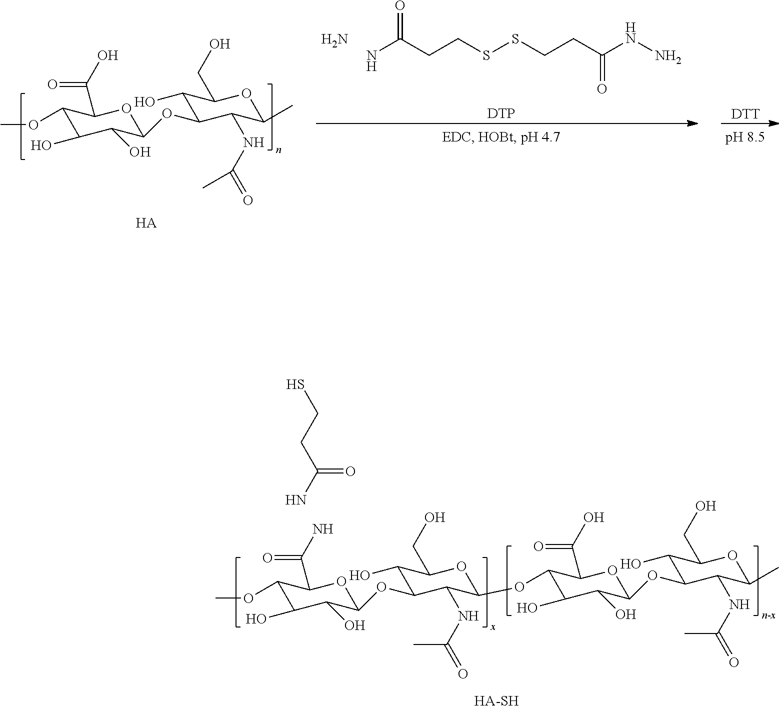

HA-SPDA was then synthesized by reacting HA-SH with SPDA in the presence of 1-[4-(2-hydroxyethoxy)-phenyl]-2-hydroxy-2-methyl-1-propane-1-one (Irgacure® 2959) with UV curing. The ¹H NMR spectra of HA-SH and HA-SPDA confirmed the synthesis of HA-SPDA with the signal peak at δ=3.0˜2.5 ppm attributed to the CH₂ groups of DTP residues and the signal peak at 2.3 ppm attributed to the CH₂ groups of SPDA residues. A schematic of the synthesis is provided.

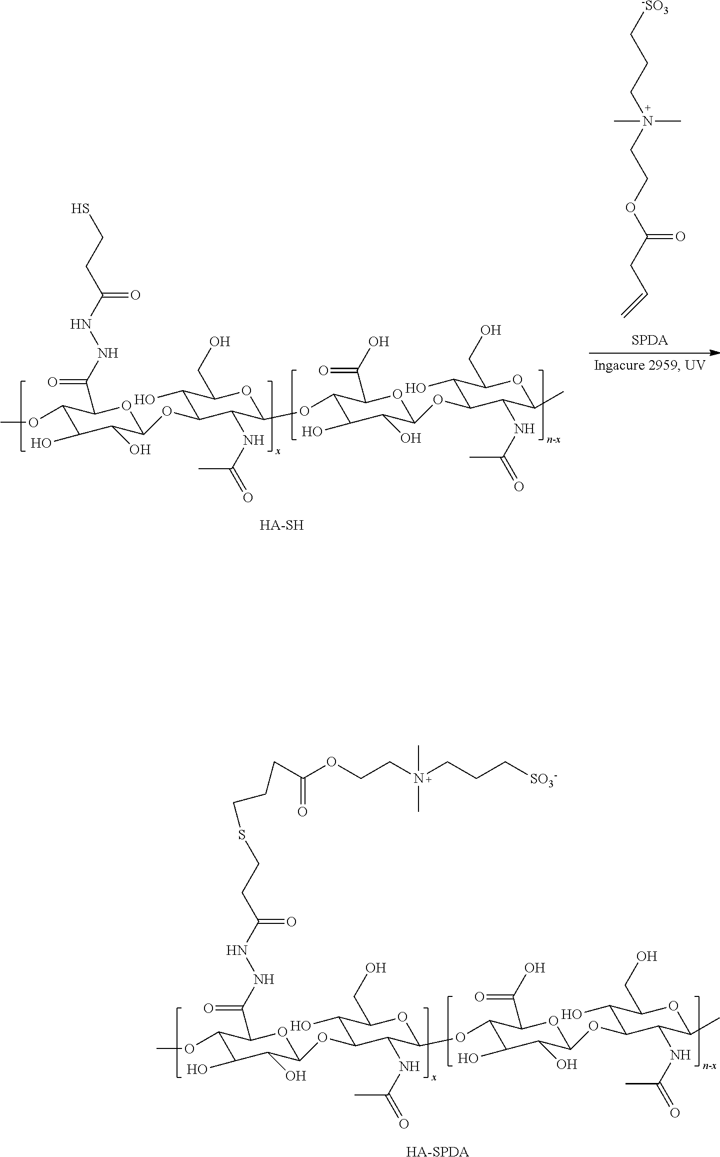

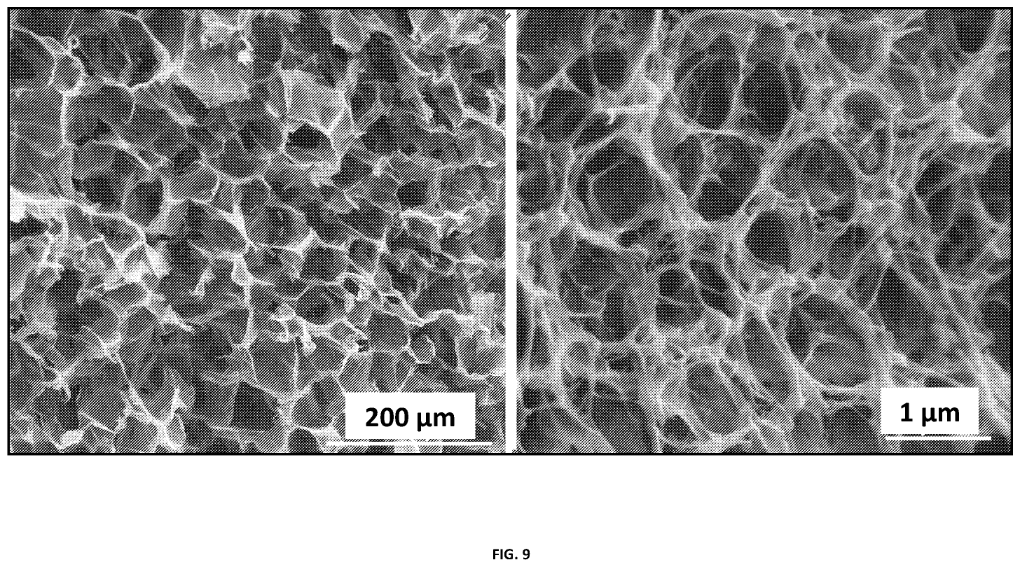

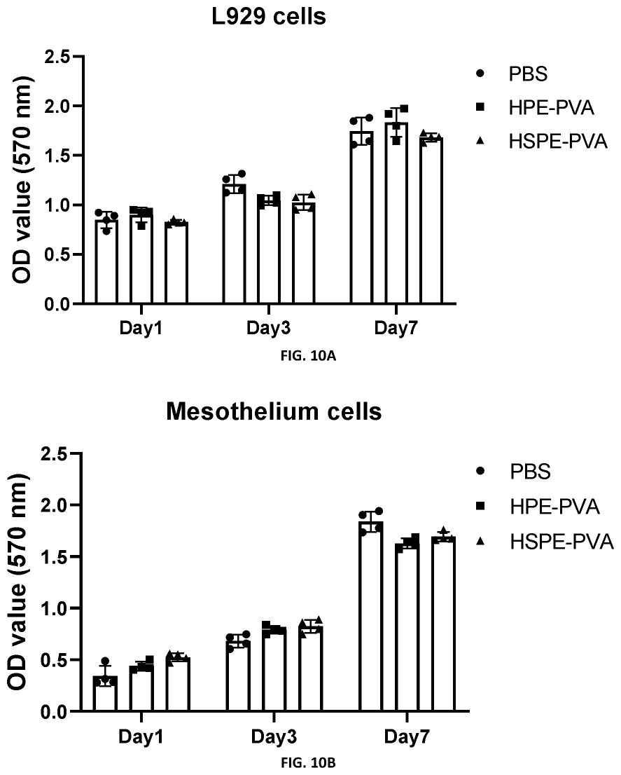

The morphology of HSPE-PVA zwitterionic hydrogel after a freeze dry was determined. FIG. 9 provides representative SEM images of the HSPE-PVA zwitterionic hydrogel showing that the hydrogels exhibited porous structures with microgel particles.

The cytotoxicity of HSPE-PVA was evaluated by a MTT assay. L929 fibroblasts were seeded into a 48-well plate at a density of 5×10³ cells/well and were cultured in DMEM containing 10% FBS in 5% CO₂. The cells were incubated with the medium with the test compound. After culturing for a series of set time points (days 1, 3, and 7), the medium was removed, and the cells were washed with PBS. About 450 μL of culture medium containing 50 μL of MTT solution (5 mg/mL in PBS) was added to each well, followed by incubation for 4 hours. Subsequently, the medium was discarded, and 500 μL of dimethylsulfoxide (DMSO) was added and shaken at 100 rpm for 30 minutes to dissolve the blue crystals in each well of the 48-well plate. Finally, the OD value was measured at 570 nm (OD570) using a microplate reader. The cells, cultured in medium containing HPE-PVA and HSPE-PVA served as treatment groups. The untreated cells incubated with the normal culture medium served as a control. The cytotoxicity of the mesothelium cells was evaluated by similar method with L929 cells. FIGS. 10A and 10B shows the in vitro cytocompatibility of HSPE-PVA zwitterionic hydrogel as the HSPE-PVA hydrogel had the same effect as HPE-PVA and PBS (untreated).

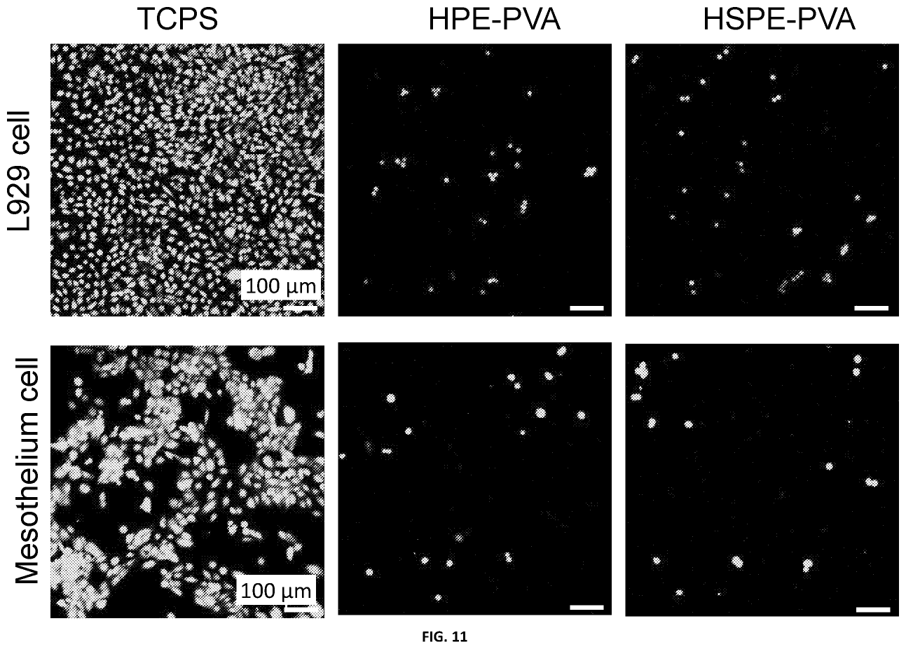

The cell adhesion on the surface of hydrogels was assessed by using murine fibroblast (L929 cells) and human mesothelium cells. The zwitterionic hydrogels were placed in a 48-well plate and incubated at 37° C. for 1 hour. L929 cells were seeded into each well at a density of 10+ cells/well and then incubated for 18 hours at 37° C. in a 5% CO₂ atmosphere. The cells were also cultured on the TCPS surface, serving as control group. After discarding the medium, the hydrogels were gently rinsed with sterile normal saline to remove the suspended cells. The morphology of L929 cells attached to the surface of hydrogels was observed at a confocal. For mesothelium cells, the protocol was similar with that of L929 cells. As seen in FIG. 11, HSPE-PVA exhibited similar antiadhesion properties as HPE-PVA.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A hydrogel comprising a carbohydrate polymer comprising carboxyl groups, a pyrogallol-containing compound, phenylboronic acid or a phenylboronic acid-containing compound, and a hydrogel forming polymer,
wherein said pyrogallol-containing compound is linked or conjugated to the phenylboronic acid or phenylboronic acid-containing compound,
wherein said a carbohydrate polymer comprising carboxyl groups is conjugated or linked to said phenylboronic acid or a phenylboronic acid-containing compound, and
wherein said a carbohydrate polymer comprising carboxyl groups is conjugated or linked to said hydrogel forming polymer via said phenylboronic acid or a phenylboronic acid-containing compound.

2. The hydrogel of claim 1, wherein said pyrogallol-containing compound is linked or conjugated to the phenylboronic acid or phenylboronic acid-containing compound via a boronic ester bond; and/or
wherein said a carbohydrate polymer comprising carboxyl groups is conjugated or linked to said hydrogel forming polymer via said phenylboronic acid or a phenylboronic acid-containing compound via a boronic ester bond.

3. The hydrogel of claim 1, wherein said a carbohydrate polymer comprising carboxyl groups is conjugated or linked to said phenylboronic acid or a phenylboronic acid-containing compound via an amide bond.

4. The hydrogel of claim 1, wherein said carbohydrate polymer comprising carboxyl groups is hyaluronic acid.

5. The hydrogel of claim 1, wherein said pyrogallol-containing compound is a flavonoid.

6. The hydrogel of claim 1, wherein said pyrogallol-containing compound is a catechin.

7. The hydrogel of claim 1, wherein said pyrogallol-containing compound is epigallocatechin gallate (EGCG).

8. The hydrogel of claim 1, wherein said phenylboronic acid-containing comprises phenylboronic acid linked to —NH₂ via a C₁-C₃ alkyl linker.

9. The hydrogel of claim 1, wherein said phenylboronic acid-containing compound is (3-aminomethylphenyl) boronic acid.

10. The hydrogel of claim 1, wherein said hydrogel forming polymer comprises polyvinyl alcohol.

11. The hydrogel of claim 1, wherein said hydrogel comprises epigallocatechin gallate, hyaluronic acid, phenylboronic acid, and polyvinyl alcohol.

12. The hydrogel of claim 11, wherein said epigallocatechin gallate is linked or conjugated to the phenylboronic acid,
wherein said hyaluronic acid is conjugated or linked to said phenylboronic acid, and
wherein said hyaluronic acid is conjugated or linked to said polyvinyl alcohol via said phenylboronic acid.

13. The hydrogel of claim 12, wherein said epigallocatechin gallate is linked or conjugated to the phenylboronic acid via a boronic ester bond;
wherein said hyaluronic acid is conjugated or linked to said polyvinyl alcohol via said phenylboronic acid via boronic ester bond; and
wherein said hyaluronic acid is conjugated or linked to said phenylboronic acid via an amide bond.

14. The hydrogel of claim 1, wherein said carbohydrate polymer comprising carboxyl groups is conjugated or linked to a zwitterionic monomer.

15. The hydrogel of claim 14, wherein said zwitterionic monomer is N, N-dimethyl-N-(2-acryloylethyl)-N-(3-sulfopropyl) ammonium betaine (SPDA).

16. The hydrogel of claim 1, wherein said hydrogel further comprises a therapeutic agent.

17. The hydrogel of claim 16, wherein said therapeutic agent is an antimicrobial, antibiotic, or anti-inflammatory.

18. The hydrogel of claim 1, wherein said hydrogel further comprises a cell.

19. A composition comprising a hydrogel of claim 1 and a pharmaceutically acceptable carrier.

20. An implantable device coated with a hydrogel of claim 1.

21. A method for inhibiting and/or preventing post-operation adhesions in a subject, said method comprising applying or administering a hydrogel of claim 1 to said subject.

22. The method of claim 21, wherein said post-operation adhesion is a post-operative internal tissue adhesion, an abdominal adhesion, a peritoneal adhesion, or a recurrent adhesion.

23. The method of claim 21, wherein said hydrogel is applied or administered to at least one injured or damaged tissue surface or to an implantable device.

24. The method of claim 21, wherein said hydrogel is applied or administered topically, by injection, by spraying, or by aerosolization.

25. A method for treating, inhibiting, and/or preventing a microbial or bacterial infection in a subject, said method comprising applying or administering a hydrogel of claim 1 to said subject, wherein said hydrogel further comprises an antimicrobial and/or antibiotic.

26. A method for treating, inhibiting, and/or preventing inflammation in a subject, said method comprising applying or administering a hydrogel of claim 1 to said subject.

27. The method of claim 26, wherein said hydrogel further comprises an anti-inflammatory.

28. A method for treating, inhibiting, and/or preventing cancer in a subject, said method comprising applying or administering a hydrogel of claim 1 to the cancer in said subject, wherein said hydrogel further comprises a chemotherapeutic agent.

29. A method for treating, inhibiting, and/or preventing diabetes in a subject, said method comprising applying or administering a hydrogel of claim 1 to said subject, wherein said hydrogel further comprises an anti-diabetic drug.

30. A method of synthesizing a hydrogel of claim 1, said method comprising mixing or combining 1) a composition comprising a complex comprising a carbohydrate polymer comprising carboxyl groups conjugated to a pyrogallol-containing compound via phenylboronic acid or a phenylboronic acid-containing compound, and 2) a composition comprising a hydrogel forming polymer.

31. The method of claim 30, wherein the compositions are combined using a 3D printer.

32. The method of claim 30, further comprising synthesizing said complex comprising a carbohydrate polymer comprising carboxyl groups conjugated to a pyrogallol-containing compound via phenylboronic acid or a phenylboronic acid-containing compound.
